SEQUENCE LISTING (6 sequences)

SEQ ID NO: 1 - DNA, 1617 bp, Artificial Sequence
  CDS (1)..(1617)
  Description of Artificial Sequence  Synthetic Primer
atggatgattttgagcgtcgcatagaacttagaaggcgaaagagggaggagatgcgactcgaagcagaaagaatcgcctaccagaggaatgacgatgatgaagaggaggcagcccgggaacggcgccgccgagcccgacaggaacggctgcggcagaagcaggaggaagaatccttgggacaggtgaccgaccaggtggaggtgaatgcccagaacagtgtgcctgacgaggaggccaagacaaccaccacaaacactcaagtggaaggggatgatgaggccgcattcctggagcgcctggctcggcgtgaggaaagacgccaaaaacgccttcaggaggctctggagcggcagaaggagttcgacccaacaataacagatgcaagtctgtcgctcccaagcagaagaatgcaaaatgacacagcagaaaatgaaactaccgagaaggaagaaaaaagtgaaagtcgccaagaaagatacgagatagaggaaacagaaacagtcaccaagtcctaccagaagaatgattggagggatgctgaagaaaacaagaaagaagacaaggaaaaggaggaggaggaagaggagaagccaaagcgagggagcattggagaaaatcagatcaaagatgaaaagattaaaaaggacaaagaacccaaagaagaagttaagagcttcatggatcgaaagaagggatttacagaagttaagtcgcagaatggagaattcatgacccacaaacttaaacatactgagaatactttcagccgccctggagggagggccagcgtggacaccaaggaggctgagggcgccccccaggtggaagccggcaaaaggctggaggagcttcgtcgtcgtcgcggggagaccgagagcgaagagttcgagaagctcaaacagaagcagcaggaggcggctttggagctggaggaactcaagaaaaagagggaggagagaaggaaggtcctggaggaggaagagcagaggaggaagcaggaggaagccgatcgaaaactcagagaggaggaagagaagaggaggctaaaggaagagattgaaaggcgaagagcagaagctgctgagaaacgccagaagatgccagaagatggcttgtcagatgacaagaaaccattcaagtgtttcactcctaaaggttcatctctcaagatagaagagcgagcagaatttttgaataagtctgtgcagaaaagcagtggtgtcaaatcgacccatcaagcagcaatagtctccaagattgacagcagactggagcagtataccagtgcagttgagggaacaaaaagcgcaaaacctacaaagccggcagcctcggatcttcctgttcctgctgaaggtgtacgcaacatcaagagtatgtgggagaaagggaatgtgttttcatcccccactgcagcaggcacaccaaataaggaaactgctggcttgaaggtaggggtttctagccgcatcaatgaatggctaactaaaaccccagatggaaacaagtcacctgctcccaaaccctctgacttgagaccaggagacgtatccagcaagcggaacctctgggaaaagcaatctgtggataaggtcacttcccccactaaggtttga

SEQ ID NO: 2 - protein, 538 aa, Artificial Sequence
  Description of Artificial Sequence  Synthetic Peptide
MDDFERRIELRRRKREEMRLEAERIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAVEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 3 - DNA, 1599 bp, Artificial Sequence
  CDS (1)..(1599)
  Description of Artificial Sequence  Synthetic Primer
atgctgggtggatccggatcgcatggaagacgcagcctggccgcgctctcccaaatcgcctaccagaggaatgacgatgatgaagaggaggcagcccgggaacggcgccgccgagcccgacaggaacggctgcggcagaagcaggaggaagaatccttgggacaggtgaccgaccaggtggaggtgaatgcccagaacagtgtgcctgacgaggaggccaagacaaccaccacaaacactcaagtggaaggggatgatgaggccgcattcctggagcgcctggctcggcgtgaggaaagacgccaaaaacgccttcaggaggctctggagcggcagaaggagttcgacccaacaataacagatgcaagtctgtcgctcccaagcagaagaatgcaaaatgacacagcagaaaatgaaactaccgagaaggaagaaaaaagtgaaagtcgccaagaaagatacgagatagaggaaacagaaacagtcaccaagtcctaccagaagaatgattggagggatgctgaagaaaacaagaaagaagacaaggaaaaggaggaggaggaagaggagaagccaaagcgagggagcattggagaaaatcagatcaaagatgaaaagattaaaaaggacaaagaacccaaagaagaagttaagagcttcatggatcgaaagaagggatttacagaagttaagtcgcagaatggagaattcatgacccacaaacttaaacatactgagaatactttcagccgccctggagggagggccagcgtggacaccaaggaggctgagggcgccccccaggtggaagccggcaaaaggctggaggagcttcgtcgtcgtcgcggggagaccgagagcgaagagttcgagaagctcaaacagaagcagcaggaggcggctttggagctggaggaactcaagaaaaagagggaggagagaaggaaggtcctggaggaggaagagcagaggaggaagcaggaggaagccgatcgaaaactcagagaggaggaagagaagaggaggctaaaggaagagattgaaaggcgaagagcagaagctgctgagaaacgccagaagatgccagaagatggcttgtcagatgacaagaaaccattcaagtgtttcactcctaaaggttcatctctcaagatagaagagcgagcagaatttttgaataagtctgtgcagaaaagcagtggtgtcaaatcgacccatcaagcagcaatagtctccaagattgacagcagactggagcagtataccagtgcaattgagggaacaaaaagcgcaaaacctacaaagccggcagcctcggatcttcctgttcctgctgaaggtgtacgcaacatcaagagtatgtgggagaaagggaatgtgttttcatcccccactgcagcaggcacaccaaataaggaaactgctggcttgaaggtaggggtttctagccgtatcaatgaatggctaactaaaaccccagatggaaacaagtcacctgctcccaaaccttctgacttgagaccaggagacgtatccagcaagcggaacctctgggaaaagcaatctgtggataaggtcacttcccccactaaggtttga

SEQ ID NO: 4 - protein, 532 aa, Artificial Sequence
  Description of Artificial Sequence  Synthetic Peptide
MLGGSGSHGRRSLAALSQIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 5 - DNA, 26 bp, Artificial Sequence
  Description of Artificial Sequence  Synthetic Primer
ggatccatggatgattttgagcgtcg

SEQ ID NO: 6 - DNA, 26 bp, Artificial Sequence
  Description of Artificial Sequence  Synthetic Primer
ggatccaaccttagtgggggaagtga

(19) United States
(12) Patent Application Publication
(10) Pub. No.: US 2005/0163755 A1
(43) Pub. Date: Jul. 28, 2005

(54) Methods and compositions related to 1-caldesmon

(76) Inventor: Alan Brent Moy, Iowa City, IA (US)

Correspondence Address: FULBRIGHT & JAWORSKI L.L.P., 600 CONGRESS AVE., SUITE 2400, AUSTIN, TX, 78701, US

(21) Appl. No.: 11/013,266

(22) Filed: Dec. 15, 2004

Related U.S. Application Data
(60) Provisional application No. 60/529,702, filed on Dec. 15, 2003.

(51) Int. Cl. A61K 48/00; C07H 21/04; C12N 15/861

(52) U.S. Cl. 424/93.2; 514/44; 435/456; 536/23.1

(57) ABSTRACT

Embodiments of the invention include compositions and methods for the inhibition of pathogen-mediated cytopathic effects by contacting a cell with an 1-CaD polynucleotide or polypeptide. In still further embodiments, a nucleic acid encoding 1-CaD is administered to cell infected by or at risk of infection by a pathogen. Gene delivery of 1-CaD shows a reduced cell toxicity compared to cytochalasin. The delivery of 1-CaD affords a protection on or therapy for modulating cell membrane integrity for protection against an infection.

This application claims priority to U.S. Provisional Patent application Ser. No. 60/529,702, filed on Dec. 15, 2003 entitled “Methods and Compositions Related to 1-Caldesmon,” which is incorporated herein by reference in its entirety. Also, the government may own rights in the present invention pursuant to grant number R01GM61732 from the National Institutes of Health.

### BACKGROUND OF THE INVENTION

I. Field of the Invention

The present invention relates generally to the fields of gene therapy and infectious diseases. More particularly, it concerns embodiments of the invention that relate to the use of gene delivery of all or part of the human actin-binding protein, 1-caldesmon, as a therapeutic agent.

II. Description of Related Art

Viral pathogens like adenovirus cause serious respiratory and gastrointestinal illness in infants, children, immunocompromised patients and military recruits. Yet, treatment for adenoviral infection is largely supportive, and there is no available antiviral therapy. It is of note that a vaccine for adenovirus has been discontinued.

Typically, the viral life cycle, as well as the life cycle of other pathogens, relies on the cell cytoskeleton. Several viruses enter, replicate and are released from infected cells by exploiting and remodeling the host cell's cytoskeleton (Bearer and Satpute-Krishnan, 2002; Jackson and Bellett, 1989; Staufenbiel et al., 1986). Viruses exploit microtubules, microfilaments and motor proteins to facilitate viral transport to the nucleus or perinuclear region for replication. Viral assembly and release during the lytic phase for viruses like herpes simplex, human immunodeficiency virus, adenovirus, and vaccinia virus are dependent on antegrade movement along microtubules and microfilaments (Bearer and Satpute-Krishnan, 2002; Leopold et al., 2000). Further, enzymes like adenoviral proteinases are activated only after it first binds to the carboxyl-terminal region on the actin cytoskeleton (Brown et al., 2002). Also, adenoviruses weaken cell membrane integrity by breaking down the actin cytoskeleton, which facilitates the release of mature virions. Thus, drugs that target the host cell's actin cytoskeleton may interfere with the viral cell cycle and represent a potential therapeutic strategy in treating viral infections.

The actin-binding protein, caldesmon (CaD) regulates actin-myosin contraction. Caldesmon exists in two forms: h-CaD, a high molecular weight form (120,000-150,000 kD) which is predominately expressed in smooth muscle and 1-CAD, a low molecular weight form (70,000-80,000 kD) which is predominately expressed in nonmuscle cells (Marston and Redwood, 1991; Matsumura and Yamashiro, 1993; Acc. No. NP_{—}149347, which is incorporated herein by reference). 1-CaD is structurally similar to h-CaD except that it lacks a central repeating region. Typically, 1-CaD is distributed along stress fibers in nonmuscle cells and is co-localized with tropomyosin (Yamashiro-Matsumura and Matsumura, 1988). Unphosphorylated 1-CaD constitutively inhibits the myosin ATPase activity (Lash et al., 1986; Tanaka et al., 1990), whereas, phosphorylated 1-CaD, under in vitro conditions, increases myosin ATPase activity through dysinhibition of 1-CaD. Furthermore, Caldesmon induces actin assembly under in vitro conditions (Galazkiewicz et al., 1989; Makuch et al., 1994).

Prior reports have examined the impact of 1-CaD overexpression in intact cells. 1-CaD is tightly regulated and is critical during mitosis and cytokinesis. 1-CaD disassociates from the actin cytoskeleton when phosphorylated by cdc2 protein kinase (Hosoya et al., 1993; Ishikawa et al., 1992; Mak et al., 1991; Yamakita et al., 1992; Yamashiro et al., 2001; Yamashiro et al., 1990). Yamashiro (2001) reported delays in cytokinesis when cells were microinjected with a 1-CaD that had mutations of all cdc2 protein kinase phosphorylation sites.

It has also been reported that overexpression of 1-CaD39, a 39 kD carboxyl-terminal fragment of wild-type 1-CaD, interfered with endogenous full-length 1-CaD function in CHO cells (Warren et al., 1994, Warren et al., 1996). These cells reportedly attached to substrate sooner and were more resistant to cytochalasin treatment than wild type cells. In contrast, they reported that overexpression of CaD40, a 40 kD amino-terminal end of CaD, did not produce a dominant negative effect (Warren et al., 1994, Warren et al., 1996). Overexpression of CaD39 has been reported to have little effect on the velocity of intracellular granule movement or the velocity and persistence of cellular translocation (Warren et al., 1996). However, CaD39-expressing cells were more elongated and encompassed less area than non-expressing cells during migration in a wound-healing assay.

Helfman (1999) has reported decreased cell contractility based on silicone wrinkling and decreased formation of focal contacts in cultured fibroblasts transiently overexpressing wild-type 1-CaD derived from human fibroblasts. Also, overexpression h-CaD in nonmuscle cells had ambiguous effects on nonmuscle cell motility (Surgucheva and Bryan, 1995).

Taken together, the prior art suggests that overexpression of wild-type 1-CaD would decrease cell adhesion, cell contractility and potentially cell motility. A decrease in cell adhesion would impair cell membrane function, cell membrane integrity and barrier function.

### SUMMARY OF THE INVENTION

The cellular cytoskeleton is critical to the viral life cycle, as well as the life cycle of other pathogens. There is a need for methods with a reduced toxicity for modulating the viral life cycle. Embodiments of the invention address this problem and others by modulating pathogen-dependent remodeling of the actin cytoskeleton. Embodiments of the invention include compositions and methods for the inhibition of pathogen-mediated cytopathic effects by contacting a cell with an 1-CaD polynucleotide or polypeptide, including a polypeptide or polynucleotide enconding a variant or derivative that maintains its ability to stabilize cellular membrane integrity, e.g., CaD39. In particular embodiments, the 1-CaD is derived from normal human endothelial cells. In still further embodiments, a nucleic acid encoding 1-CaD is administered to cell infected by or at risk of infection by a pathogen. Gene delivery of 1-CaD shows a reduced cell toxicity compared to cytochalasin. This invention demonstrates that delivery of 1-CaD affords a protection on or therapy for modulating cell membrane integrity for protection against an infection. One unique aspect of the invention is that low transfection efficiency of the 1-CaD gene is effective in stabilizing cellular membrane integrity in the face of a competing adenovirus infection through a mechanism independent of actin assembly and myosin ATPase activity. Additionally, 1-CaD expression attenuated adenovirus-mediated effects with less cell toxicity than cytochalasin.

Further embodiments of the invention include pharmaceutical compositions comprising a nucleic acid encoding 1-CaD or a variant thereof in a pharmaceutically acceptable carrier. In certain embodiments, the nucleic acid is an expression cassette. The expression cassette may be included in an expression vector. The expression vector can be a mammalian expression vector and in certain embodiments the expression vector is a viral expression vector. The expression vector can be an adenoviral, an adenoviral associated virus, a lentiviral, a HSV, a MMLV, a vaccinia vector or other expression vector known to one of skill in the art. In particular aspects, the expression vector is an adenoviral or lentiviral expression vector. The adenoviral expression vector can be a replication-competent, a conditionally replication-competent or a replication-defective adenovirus. In other aspects, the adenoviral expression vector may lack all or part of an E1 coding sequence. Furthermore, the expression vector may encode or the delivery vector may be modified so as to impart an altered tropism to the delivery vector. In various embodiments, the mammalian viral expression vector encodes at least a second therapeutic protein. Compositions of the invention may comprise at least 1, 2, 3, 4, 5 or more pharmaceutically acceptable excipients. In various embodiments, the composition is capable of being nebulized. Compositions of the invention may be delivered by a variety of means including, but not limited to, inhalation, injection, topical administration, or ingestion.

Further aspects of the invention include an isolated polynucleotide comprising a nucleic acid sequence encoding all or part of a 1-CaD polypeptide, or derivative or variant thereof that stabilizes cellular membrane integrity. The isolated polynucleotide may encode a 1-CaD polypeptide as set forth in SEQ ID NO:2 or SEQ ID NO:4. The polynucleotide may further include a promoter sequence and/or a polyadenylation signal. In certain embodiments, the polynucleotide is comprised in a viral vector, in particular an adenoviral vector. The adenoviral vector may encode a replication-competent, a conditionally replication-competent or a replication-defective adenovirus.

Still further embodiments include methods comprising administering an effective amount of an expression vector or an expression cassette encoding 1-CaD to a subject infected with or at risk of being infected with a pathogen. The pathogen may be a viral pathogen such as adenovirus. In one embodiment, the expression vector is an adenoviral vector. The adenoviral vector may be administered as approximately 10² to 10¹⁴ plaque forming units of adenovirus/kg body weight. The expression vector may be administered as a single or multiple doses. Multiple doses includes 1, 2, 3, 4, 5, 6, 7, 8, 9, 10 or more doses. Administration of the expression vector may be by inhalation, ingestion, injection or topical administration.

Other embodiments of the invention include methods for attenuating a pathogen infection, such as a viral infection, by administering to a subject having or at risk of having an infection an effective amount of an expression cassette encoding 1-CaD or a derivative thereof. In particular embodiments, the subject is immunocompromised. The expression cassette may be comprised in a viral vector. In certain embodiments, the viral vector is an adenoviral vector. The methods may further comprising administrating at least a second antiviral composition. The second antiviral composition may be interferon, nucleoside analogs, cytosine-arabinoside, adenine-arabinoside, iodoxyuridine, acyclovir or other known antivirals. Compositions of the invention may be administered by inhalation, ingestion, injection or topical administration. In certain aspects, the viral infection or adenoviral infection is a respiratory infection.

In still further embodiments, a prophylactic method includes administering an effective amount of expression vector encoding 1-CaD or a derivative thereof to a subject at risk of infection by a pathogen. The expression vector may be an adenoviral expression vector. The vector is administered by inhalation, ingestion, injection or topical administration. The pathogen may be a viral pathogen, such as adenovirus. A viral expression vector may be administered at a dose of approximately 10², 10³, 10⁴, 10⁵, 10⁶, 10⁷, 10⁸, 10⁹, 10¹⁰, 10¹¹, 10¹², 10¹³, to 10¹⁴ plaque forming units of adenovirus/kg body weight, or any value or range there between. In various embodiments, an expression vector can be administered in a single or multiple doses.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. FIG. 1A is a western blot using an antibody directed against 1-CaD in cell lysate from DF-1 cells. Lanes 1-4 represents increasing amount of DF-1 lysate: (1) 5 μl of cell lysate; (2) 10 μl of cell lysate; (3) 15 μl of cell lysate; (4) 20 μl of cell lysate. Lane 5 represents 15 μl of cell lysate from cultured PPAEC; lane 6 represents 20 μl of PPAEC lysate. FIG. 1B shows the analysis of a RT-PCR of isolated total RNA of cultured DF-1 cells showing null expression for 1-CaD mRNA expression. Lane 1 and 2 show DNA molecular weight markers. Lane 3 shows the 323 bp product of beta actin, lanes 4 shows no 1-CaD product in DF-1 cells, Lane 5 shows the 1.6 Kb 1-CaD cDNA of cultured PPAEC. FIG. 1C shows an analysis of a RT-PCR of isolated total RNA from cultured HUVEC (lane labeled “H”) and cultured PPAEC (lane labeled “P”). The analysis shows a full-length cDNA expression of 1-CaD, while lane labeled “D” represents no PCR product in DF-1 cells.

FIG. 2. A Western blot showing dose-dependent protein expression of heterologous human 1-CaD in DF-1 cells transfected at different multiplicities of infection (MOIs) by an adenovirus encoding 1-CaD (Ad-1-CaD) using a Calcium phosphate (CaPi) co-precipitation procedure as described below.

FIGS. 3A-3C. Co-localization of human 1-CaD with avian microfilaments in DF-1 cells determined by immunofluorescence. Cells were co-stained with Texas Red-phalloidin and Oregon Green conjugated goat anti-mouse secondary antibody and mouse anti-1-CaD primary antibody to visualize the microfilaments and 1-CaD, respectively. Images were taken using FITC and rhodamine filters. Images depicting 1-CaD stained cells (FIG. 3A) and actin stained cells (FIG. 3B) from the same field of view were then overlayed (FIG. 3C) to show co-localization, which demonstrated white filaments or lighter saturation of actin filaments.

FIG. 4. Transfection efficiency of DF-1 cells defined by the fraction of cells that exhibit 1-CaD-decorated microfilaments, based on Oregon Green labeling of 1-CaD, to the total number of cells that express microfilaments based on Texas Red labeling of F-actin. Several fields of view were used and averaged to attain total efficiency for each MOI. Data represents the average and standard error of 100 or more counted cells for each MOI.

FIG. 5. A representative experiment of fibroblast cell attachment dynamically quantitated by the measured transcellular resistance in a confluent monolayer. Transcellular resistance was measured in confluent cultured fibroblast monolayers grown on a gold microelectrode by applying an alternating current. A confluent density of viable cells, based on trypan blue exclusion, was inoculated on the biosensor. Transcellular resistance increased rapidly within 2 hrs and achieved a steady state level after 15 hrs when cells were fully spread.

FIG. 6. Transcellular resistance comparison of DF-1 cells transfected with Ad-1-CaD and cells transfected with Ad only at 15 hrs of attachment at MOI doses between 10-500. Resistance values are expressed as final resistance (15 hr) minus initial resistance (0 hr) and normalized to the resistance of untransfected wild type DF-1 cells. The expression of 1-CaD mediates an attenuated dose-dependent loss in transcellular resistance in cells transfected with controlled Ad virus. Data represents the average and standard error for each MOI. Sample size was >15 for each condition. “*” denotes a statistically significant difference compared to cells receiving the controlled virus based on a student t-testing (p<0.05).

FIG. 7. The front panel of the software program that provides a best fit of the calculated real and imaginary values to the experimental transcellular real and imaginary data across a cell-covered electrode as a function of electrical frequency between 22 to 60,000 Hz. The adjustable vertical bars limit the curve fitting process to the frequency range (5,000-60,000 Hz) even though the calculated real and imaginary values are displayed over a frequency between 22-60,000 Hz. Calculated real and imaginary values were derived from a numerical model that depends on the impedance from cell-cell adhesion (Rb), cell-matrix adhesion (α) and membrane capacitance (Cm). Calculated real and imaginary measurements were determined and plotted from the solutions of α, Rb and Cm obtained from an iterative multi-response Levenberg-Maquardt non-linear optimization algorithm to search for the best fit to the experimental data.

FIGS. 8A-8D. FIG. 8A shows a comparison of the difference in α, Rb, and Cm between wild-type DF-1 cells, cells transfected with Ad-1-CaD and cells transfected with controlled Ad construct at 200 MOI. Data were statistically analyzed by an analysis of variance on a Tukey multiple comparison of the group means. Data marked by a “*” represents a statistically significant comparison (p<0.05) against the wild-type data. Data marked by a “**” represents a statistically significant comparison (p<0.05) between cells transfected with Ad-1-CaD and cells transfected with controlled virus. Each data represents the mean (±SE) with an n>10. FIG. 8B compares the effect between 3 μM cytochalasin D and 200 MOI of Ad-CaD or controlled Ad on transcellular resistance in DF-1 monolayers. Resistance is normalized to wild-type cell resistance. Data marked by “*” represents a statistically significant change compared to cells exposed to Ad-1-CaD. FIG. 8C illustrates the impact of 3 μM cytochalasin D on α, Rb, and Cm in wild-type DF-1 cells. Each data represents the mean (±SE) with an n>13. See below for explanation. FIG. 8D compares the effect of 3 μM cytochalasin D, 200 MOI Ad-1-CaD and 200 MOI controlled Ad on cell viability measured by trypan blue staining after a period of 48 hrs. Data marked by “*” represents a statistically significant change compared to cells exposed to controlled Ad (p<0.05).

FIGS. 9A-9D. Illustrates immunofluorescent images by exposing fixed and permeabilized cells to a primary antibody against 1-CaD and a secondary antibody conjugated with Oregon Green (FIG. 9A and FIG. 9C). IRM images were taken of the same corresponding cells to evaluate cell-substrate contact (FIG. 9B and FIG. 9D). Images were taken of cells transfected with Ad-empty (FIG. 9A and FIG. 9B) and Ad-1-CaD (FIG. 9C and FIG. 9D) at MOI 200. Areas of the cell that are in close contact with the substrate appear dark. The arrowheads in the IRM images indicate the cell-matrix interaction of those cells that express 1-CaD.

FIG. 10. DF-1 cells cultured on glass grid etched coverslips and wounded with a pipette tip. The dotted line represents the edge of the wound. Ad-1-CaD and Ad-empty transfected cells are in the left and right columns, respectively. Three measurements were taken at equidistant points and averaged from each time point to determine cell velocity.

FIG. 11. Cell motility (μm/hr) comparison between DF-1 cells transfected with Ad-1-CaD and Ad-empty at 200 and 500 MOI. Data shows no significant differences in cell motility at either MOI dose. The data represent the means and standard error of three replicate experiments. Each time point represents an n=3. The analysis was repeated three times.

FIG. 12. Cells cultured on a collagen membrane poured between 2 polyethylene bars with Velcro strips attached. Polyethylene bars are attached to a force transducer. Tension readings are then taken from each force transducer, filtered, and recorded on the computer using a program written in LabView. The length-tension properties were determined to isolate the length of the collagen lattice for a no-load state. The initial length (l₀) for a no-load state was first determined. FIG. 12 illustrates a representative experiment in which 3-μM cytochalasin-D abolishes isometric tension in cultured DF-1 cells. Constitutive tension is defined by the amount of cytochalasin D-mediated tension loss.

FIG. 13. The amount of prestress loss upon exposure to cytochalasin D in cells transfected with either Ad-1-CaD or Ad-empty at 200 and 500 MOI. FIG. 13 shows the average change in force from baseline, sham and upon exposure to 3 μM cytochalasin-D. No significant difference in constitutive tension was seen between Ad-1-CaD and Ad-empty transfected cells. There was no significant difference in prestress reduction by increasing 1-CaD expression. The data is expressed as the means and standard errors for 10 or more force measurements.

FIG. 14. Comparison of the amount of actin assembly between wild-type cells, cells transfected with the control virus and cells transfected with Ad-1-CaD. Cells were lysed with a Triton-X buffer and soluble and insoluble cytoskeletal fractions were collected, separated by SDS-PAGE and measured using a calibration standard of purified nonmuscle actin. Only the insoluble actin fraction is displayed. Data marked by an “*” represents a statistically significant reduction in insoluble actin fraction compared to wild-type cells (p<0.05).

FIG. 15. All mutants have been confirmed by protein expression and nucleic acid sequencing and actin localization.

FIG. 16. Illustrates the results from transfection of wt-CaD that showed no significant difference in TER in cultured PPAEC compared to monolayers that were transfected with the controlled virus

FIG. 17. Figure compares differences in constitutive PPAEC resistance in cells transfected with lentiviral mocked construct that encode for the CAT gene; constructs that contain the gene that encode for the full-length wt CaD (Fl-CaD), and construct that contains a CaD mutant in which the serine 504 and 534 have been mutated to glutamic acid (CaD504/534).

FIG. 18. Figure illustrate the effect of phorbol ester (PDBU) on TER in cultured PPAEC that have been mock transfected with the CAT gene; transfected with the lentiviral wt-CaD construct; and the lentiviral CaD S504/534E construct. There is no difference in barrier function in response to PDBU stimulation.

FIG. 19. Comparison of transfecting lentiviral constructs on TER in cultured Df-1 cells mocked transfected with CAT gene (CAT); transfected with gene that encodes for the full-length wt CaD (Fl-CaD); and transfected with a construct that encodes for CaDS504/534E.

FIG. 20. Figure depicts mean changes in fractional TER in thrombin-exposed monolayers that have been mocked transfected; transfected with wt-CaD construct; and transfected with the S504/534E construct. Expression of the S504/534E decreased TER and impeded recovery, while expression of wt-CaD only impeded recovery.

FIG. 21. Figure depicts the mean constitutive TER in monolayers that are transfected with the mock construct, wt-CaD construct and the CaD39 construct. There was no observed difference in constitutive TER between mock and wt-CaD transfected cells. Yet, cells transfected with CaD39 displayed significantly higher TER.

FIG. 22. Figure depicts mean fractional change in TER in thrombin-exposed monolayers that were transfected with the CAT construct, wt CaD construct and CaD39 construct. Wt-CaD attenuated the restoration, while CaD39 attenuated both the decline and the restoration of thrombin-mediated barrier dysfunction.

FIG. 23. Figure depicts mean fractional changes in TER in PDBU-stimulated PPAEC that have been transfected with CAT construct, wt CaD construct and the CaD39 construct. Expression of CaD39 attenuated the rate and amplitude of decline in TER in PDBU-stimulated monolayers.

### DESCRIPTION OF ILLUSTRATIVE EMBODIMENTS

The cellular cytoskeleton is critical to the viral life cycle, as well as the life cycle of other pathogens. Agents like cytochalasin inhibit viral infections, but cannot be used for antiviral therapy because of their toxicity, see below. Therefore, there is a need for less toxic strategies for modulating the viral life cycle. Embodiments of the invention address this problem and others by modulating pathogen-dependent remodeling of the actin cytoskeleton. Various embodiments of the invention demonstrate an effective, safe compositions and methods by which expression of 1-CaD can protect cell-membrane integrity and abrogate or modulate infection by a pathogen, e.g., adenovirus infection. In one embodiment, gene delivery of human 1-CaD attenuates high dose adenovirus-mediated loss in transcellular resistance at low transfection efficiency. This protection is due to 1-CaD's effect on membrane capacitance and is mediated independent of actin assembly or myosin ATPase activity. Expression of human 1-CaD exhibits less cell toxicity as measured by trypan blue exclusion than cytochalasin and, unlike cytochalasin, it does not interfere with wound closure or adversely effect transcellular resistance. This invention demonstrates that the expression of 1-CaD as a potentially efficacious and safe agent for inhibiting various cytopathic effects of pathogens, such as adenovirus, without significantly interfering with general cellular function. Thus, expression of 1-CaD can be a more efficient targeted therapy to prevent viruses from exploiting the actin cytoskeleton and myosin motors.

The actin cytoskeleton regulates a diverse series of housekeeping functions like mitosis, cytokinesis, contraction, cell motility, wound repair and barrier function (Belisle and Abo, 2000; Coomber, 1991; Eberle et al., 1990; Eckes et al., 2000; Fishkind et al., 1991; Gandhi et al., 1997; Grinnell, 1994; Hecht et al., 1996; Holwell et al., 1997; Imaizumi et al., 1996; Montesano and Orci, 1988; Moy et al., 2002; Moy et al., 1998; Moy et al., 1996; Pilcher et al., 1995; Schuppan et al., 1995; Warren et al., 1996). Pharmacological remodeling of the actin cytoskeleton alters the virulence of several viruses. Cytochalasin, a fungal metabolite that acts as a potent inhibitor of actin filament and contractile microfilaments, inhibits viral infections under in vitro conditions (Elliott and O'Hare, 1997; Iyengar et al., 1998; Li et al., 1998; van Loo et al., 2001). However, cytochalasin causes significant cell toxicity that prevents its clinical use as an antiviral agent.

Motor proteins for regulating actin-myosin contraction in smooth muscle and nonmuscle cells are dependent on phosphorylation of the regulatory myosin light chain (MLC). Phosphorylation of MLC in smooth muscle and nonmuscle cells is regulated by calcium-calmodulin activation of myosin light chain kinase (MLCK), which increases the actin-myosin ATPase activity (Adelstein, 1978; Adelstein and Conti, 1975; Aksoy et al., 1983; Daniel et al., 1981; Moy et al., 1993). Additionally, the GTPase Rho also regulates phosphorylation of MLC by activating Rho-kinase, which, in turn, phosphorylates MLC (Amano et al., 1996). GTPase Rho also phosphorylates the myosin-binding subunit of MLC phosphatase, which inhibits MLC dephosphorylation (Kimura et al., 1996). A decrease in steady-state dephosphorylation leads to unopposed MLC phosphorylation, which increases myosin ATPase activity. MLC is phosphorylated by other kinases (Kawamoto et al., 1989; Singer, 1990) that add to the redundancy of signaling pathways that activate myosin motors. Since there is a redundancy of upstream signaling pathways that activate myosin motors, therapies that interfere with these pathways may represent an ineffective strategy to interfere with the viral life cycle and may result in toxic side effects.

The efficacy and safety of the prophylactic and/or therapeutic expression of 1-CaD is exemplified using DF-1 cells, a spontaneously immortalized but non-transformed avian fibroblast line that is null for 1-CaD. As a 1-CaD knockout cell line, use of DF-1 cells permits accurate quantification of transfection efficiency. Heterologous human wild-type 1-CaD was transiently expressed and localized with avian microfilaments in DF-1 cells using a replication-deficient adenovirus expression system. A number of quantitative and dynamic bioengineering assays are used to evaluate a variety of cellular functions. These assays used in conjunction with 1-CaD expression identify novel methods of use for 1-CaD. In particular, cytoskeletal-membrane properties were evaluated by measuring transcellular impedance across a cultured cell monolayer grown on a microelectrode sensor. A minute alternating current was applied across the culture monolayer, and three defined current paths in this electrical circuit were quantified, which reflect measurements of cell-cell and cell-matrix adhesion and membrane capacitance, a property of membrane folding (Moy et al., 2002, Moy et al., 2000).

III. Caldesmon

Caldesmon (CaD) is an actomyosin regulatory protein found in smooth muscle and nonmuscle cells. Domain mapping and physical studies suggest that CaD is an elongated molecule with an N-terminal myosin/calmodulin-binding domain and a C-terminal tropomyosin/actin/calmodulin-binding domain. Humphrey et al. (1992) used a probe encoding part of avian caldesmon to screen a human aorta library and clone smooth-muscle and nonmuscle CaD-encoding cDNAs. The predicted smooth-muscle polypeptide is 793 amino acids long. As in the case of chicken CaD, non-muscle CaD was missing a central helical domain of 256 amino acids. The non-muscle form appears to be generated by exon skipping.

A high molecular weight caldesmon (h-CaD) is predominantly expressed in smooth muscles, whereas the low molecular weight caldesmon (1-CaD) is widely distributed in nonmuscle tissues and cells. Hayashi et al. (1992) demonstrated that the human CaD gene is composed of 14 exons. Fluorescence in situ hybridization (FISH), showed that it is encoded by a single gene located at 7q33-q34. For a review of CaD see Sobue and Sellers (1991) and Huber (1997).

A process of cloning 1-caldesmon from Hela cells by screening a cDNA library and generating oligonucleotides to the calmodulin and actin-binding domains has been described in U.S. Pat. Nos. 5,532,337 and 5,739,088, which are incorporated herein by reference. When expressed in bacteria, these polynucleotides encode for polypeptides that confer calmodulin and actin-binding activity under in vitro conditions.

A. Caldesmon Polynucleotides

Certain embodiments of the present invention concern nucleic acids encoding caldesmon, in particular, nucleic acid sequence as set forth in SEQ ID NO:1, or SEQ ID NO:3. Various nucleic acid sequences deposited with Genbank are related to CaD and may be used in the methods described herein, such as accession numbers M64110 (gi179829), D90452 (gi219895), D90453 (gi219897), M83216 (gi306508), AF247820 (gi13186200), BC005006 (gi14709703), BC015839 (gi16198382), BC040354, (gi25955665), and BC014035 (gi33878449), which are incorporated herein by reference. In certain aspects, both wild-type and mutant versions of these sequences are employed. In particular aspects, a nucleic acid comprises a nucleic acid segment of SEQ ID NO:1 or SEQ ID NO:3 or a biologically functional equivalent thereof.

The term “nucleic acid” is well known in the art. A “nucleic acid” as used herein will generally refer to a molecule (i.e., a strand) of DNA, RNA or a derivative or analog thereof, comprising a nucleotide base. A nucleotide base includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleotide bases in length. The term “polynucleotide” refers to at least one molecule of greater than about 100 nucleotide bases in length.

In certain embodiments, a “gene” refers to a nucleic acid that is transcribed. In certain aspects, the gene includes regulatory sequences involved in transcription or message production. In particular embodiments, a gene comprises transcribed sequences that encode for a protein, polypeptide or peptide. As will be understood by those in the art, this functional term “gene” includes genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered nucleic acid segments may express, or may be adapted to express proteins, polypeptides, polypeptide domains, peptides, fusion proteins, mutant polypeptides and/or the like.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid as originally isolated, and does not exclude genes or coding regions later added to the nucleic acid by the hand of man.

B. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. Various mechanisms of oligonucleotide synthesis may be used, such as those methods disclosed in, U.S. Pat. Nos. 4,659,774; 4,816,571; 5,141,813; 5,264,566; 4,959,463; 5,428,148; 5,554,744; 5,574,146; 5,602,244 each of which are incorporated herein by reference.

A non-limiting example of an enzymatically produced nucleic acid include nucleic acids produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

C. Purification of Nucleic Acids

A nucleic acid may be purified on polyacrylamide gels, cesium chloride centrifugation gradients, column chromatography or by any other means known to one of ordinary skill in the art (see for example, Sambrook et al., 2001, incorporated herein by reference).

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells.

D. Nucleic Acid Segments

In certain embodiments, the nucleic acid is a nucleic acid segment. As used herein, the term “nucleic acid segment,” are smaller fragments of a nucleic acid, including, but not limited to those nucleic acids encoding only part of SEQ ID NO:1, SEQ ID NO:3 or referenced herein. Thus, a “nucleic acid segment” may comprise any part of a gene sequence, of from about 8 nucleotides to the full length of SEQ ID NO:1, SEQ ID NO:3 or other sequences referenced herein.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm may be applied to each of SEQ ID NO:1 or SEQ ID NO:3. As used herein, a “probe” generally refers to a nucleic acid used in a detection method or composition. As used herein, a “primer” generally refers to a nucleic acid used in an extension or amplification method or composition.

In a non-limiting example, one or more nucleic acid constructs may be prepared that include a contiguous stretch of nucleotides identical to or complementary to SEQ ID NO:1 or SEQ ID NO:3. A nucleic acid construct may be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 60, about 70, about 80, about 90, about 100, about 200, about 500, about 1,000, about 2,000, about 3,000, about 5,000, about 10,000, about 15,000, to about 20,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values). Non-limiting examples of intermediate lengths include about 11, about 12, about 13, about 14, about 15, about 16, about 17, about 18, about 19, about, 20, about 21, about 22, about 23, about 24, about 25, about 26, about 27, about 28, about 29, about 30, about 35, about 40, about 50, about 60, about 70, about 80, about 90, about 100, about 125, about 150, about 175, about 200, about 500, about 1,000, to about 10,000 or more bases.

The term “functionally equivalent codon” is used herein to refer to codons that encode the same amino acid, such as the six codons for arginine and serine, and also refers to codons that encode biologically equivalent amino acids. Thus, the most preferred codon for alanine is thus “GCC”, and the least is “GCG.” Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:1 or SEQ ID NO:3 will be nucleic acid sequences that are “essentially as set forth in SEQ ID NO:1 or SEQ ID NO:3.

IV. Caldesmon Gene Therapy

In other embodiments, it is envisioned that nucleic acids encoding prophylactic or therapeutic polypeptides or peptides of the invention may be utilized in gene therapy. Individuals who are exposed to or are at risk of exposure to pathogens that utilize the cellular architecture as a component of their life cycle may be the subject of gene therapy in which nucleic acids encoding for 1-CaD polypeptides or derivatives thereof are incorporated into host cells. To facilitate gene therapy, the cDNA for 1-CaD polypeptides or derivatives thereof can be incorporated into an expression construct or an expression cassette for delivery to a target cell or cell population.

Expression typically requires that appropriate signals be provided in the vectors or expression cassettes, and which include various regulatory elements, such as enhancers/promoters from viral and/or mammalian sources that drive expression of the genes of interest in host cells. Elements designed to optimize messenger RNA stability and translatability in host cells may also be included. Drug selection markers may be incorporated for establishing permanent, stable cell clones.

Viral vectors are preferred eukaryotic expression systems. Included are adenoviruses, adeno-associated viruses, retroviruses, herpesviruses, lentivirus and poxviruses including vaccinia viruses and papilloma viruses including SV40. Viral vectors may be replication defective, conditionally defective or replication competent.

By using bioengineering tools that quantify cytoskeletal-membrane properties, this invention demonstrates the efficacy and safety of gene delivery of 1-CaD to inhibit the cytopathic effects of adenovirus. Gene delivery of human wild-type 1-CaD inhibits the cytopathic effects of adenovirus at low transfection efficiency with much less cell toxicity than cytochalasin. This efficacy was documented with only 20-25 percent transfection efficiency.

Gene delivery of 1-CaD abrogated adenovirus-mediated loss in transcellular resistance, which is a measurement of cell-membrane integrity. Using a mathematical model of the experimental transcellular impedance, expression of 1-CaD had the most impact on transcellular resistance by affecting membrane capacitance; resulting in a value that was not statistically different from wild-type cells but statistically different from cells exposed to controlled virus. The effect of 1-CaD on membrane capacitance suggests that 1-CaD stabilized the cortical actin cytoskeleton, which, in turn, stabilized membrane convolution. Expression of 1-CaD had modest effects on stabilizing cell-cell adhesion in the presence of adenovirus. Expression of 1-CaD had an intermediate enhancing effect on cell-matrix adhesion in response to exposure from adenovirus.

Gene delivery of 1-CaD does not adversely affect static and dynamic cellular housekeeping functions as does cytochalasin. Expression of wild-type 1-CaD does not have any significant impact on wound closure in response to mechanical wounding. There is no statistical difference in cell velocity upon wound closure between cells expressing 1-CaD and controlled cells. This behavior was consistent even at higher MOI dosages of Ad-CaD. In contrast, cytochalasin D completely prevents wound repair.

Various embodiments of the invention demonstrate that gene delivery of 1-caldesmon protected adenovirus-mediated cell injury independent of effects on contractility or through effects on actin assembly, which was not predicted from prior art reported under in vitro conditions.

Gene delivery of 1-CaD also caused less cell toxicity based on trypan blue dye exclusion than that observed in response to cytochalasin during the same exposure period. Our invention demonstrates that gene delivery of 1-CaD protects cell membrane integrity in face of an adenoviral infection with minimal impact on cell toxicity and housekeeping functions such as wound repair and cell adhesion.

A. Vectors and Expression Constructs

The term “vector” is used to refer to a carrier nucleic acid molecule into which a nucleic acid sequence can be inserted for introduction into a cell where it can be replicated and/or expressed. A nucleic acid sequence can be “exogenous” or “heterologous” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well, as described below.

In order to express a 1-CaD peptide or polypeptide or a derivative thereof it is necessary to provide a 1-CaD gene in an expression vehicle. The appropriate nucleic acid can be inserted into an expression vector by standard subcloning techniques. The manipulation of these vectors is well known in the art. Examples of fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.). The vector carrying the gene of interest is transfected into Spodoptera frugiperda (Sf9) cells by standard protocols, and the cells are cultured and processed to produce the recombinant protein. Mammalian cells exposed to baculoviruses become infected and may express the foreign gene only. This way one can transduce all cells and express the gene in dose dependent manner.

There also are a variety of eukaryotic vectors that provide a suitable vehicle in which recombinant polypeptide can be produced. HSV has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 1 and 2 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct. Use of the baculovirus system will involve high level expression from the powerful polyhedron promoter.

TABLE 1
PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α_{1-Antitrypsin}
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 2

| Element | Inducer |
|---|---|
| MT II | Phorbol Ester (TPA) |
| | Heavy metals |
| MMTV (mouse mammary tumor | Glucocorticoids |
| virus) | |
| β-Interferon | Poly(rI)X |
| | Poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

1. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

a. Adenoviral Vectors

A particular method for delivery of the nucleic acid involves the use of an adenovirus expression vector, which can be replication defective, conditionally replication competent or replication competent. Exemplary adenovirus compositions and methods can be found in U.S. Pat. Nos. 6,638,502, 6,602,706, 6,630,574, each of which is incorporated herein by reference. Although adenovirus vectors are known to have a low capacity for integration into genomic DNA, and in addition, demonstrate high efficiency of gene transfer. “Adenovirus expression vector” is meant to include those constructs containing adenovirus sequences sufficient to (a) support packaging of the construct and (b) to ultimately express a construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the methods of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

C. Retroviral Vectors

Retroviruses have promise as therapeutic vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding a 1-CaD or derivative thereof) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

e. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989; Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, the host cell or tissue may be comprised in at least one organism. In certain embodiments, the organism may be, but is not limited to, a prokaryote (e.g., a eubacteria, an archaea), an eukaryote, a patient or a subject, as would be understood by one of ordinary skill in the art (see, for example, webpage http://phylogeny.arizona.-edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using all or part of SEQ ID NO: 1 or SEQ ID NO:3.

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein, polypeptide and peptide sequences for various CaD and 1-CaD genes have been previously disclosed, and may be found at computerized databases known to those of ordinary skill in the art. One such database is the National Center for Biotechnology Information's Genbank and GenPept databases (www.ncbi._nlm.nih.gov/). The coding regions for these known genes may be amplified and/or expressed using the techniques disclosed herein or by any technique that would be known to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

B. Selectable Markers

In certain embodiments of the invention, a cell may contain a nucleic acid construct of the present invention and may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. e.g., 1-CaD or a derivative thereof. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

D. Combination Therapies

In order to increase the efficacy of a caldesmon therapy, it may be desirable to combine more than one therapeutic approach in the treatment of disease. More generally, these other compositions would be provided in a combined amount effective to kill or inhibit the growth of a pathogen or an abnormal cell. This process may involve subjecting the subject to both therapies at the same time. Alternatively, one therapy may precede or follow the other therapy by intervals ranging from minutes to weeks. Generally, one would ensure that a significant period of time did not expire between the time of each therapy such that both therapies would still be able to exert an advantageously combined effect on the target pathogen or cell. In such instances, it is contemplated that one may contact a target cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Various combinations may be employed, where the caldesmon therapy is “A” and the secondary agent, such as anti-pathogenic agent, such as an antiviral or antimicrobial is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

V. Caldesmon Proteinaceous Compositions

In certain embodiments, the present invention concerns compositions comprising at least one proteinaceous molecule. The proteinaceous molecule may be 1-CaD or a derivative thereof. The proteinaceous molecule may also be used, for example, in a pharmaceutical composition for the delivery of a therapeutic agent or as part of a screening assay to identify modulators or the cytoskeleton and cellular architecture. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments, the size of the at least one proteinaceous molecule may comprise, but is not limited to, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 or greater amino molecule residues, and any range derivable therein. Furthermore, such proteinaceous molecules may include 10, 20, 30, 40, 50, 60, 70, 80, 90, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750 or more contiguous amino acid residues from SEQ ID NO:2 or SEQ ID NO:4.

Accordingly, the term “proteinaceous composition” encompasses amino molecule sequences comprising at least one of the 20 common amino acids in naturally synthesized proteins, or at least one modified or unusual amino acid.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance, which produces no significant untoward effects when applied to, or administered to, a given organism according to the methods and amounts described herein. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that it will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

A. Isolating Proteinaceous Compounds

1-CaD or derivatives thereof may be obtained according to various standard methodologies that are known to those of skill in the art. For example, antibodies specific for 1-CaD may be used in immunoaffinity protocols to isolate the respective polypeptide from infected cells, in particular, from infected cell lysates. Antibodies are advantageously bound to supports, such as columns or beads, and the immobilized antibodies can be used to pull the 1-CaD target out of the cell lysate.

Alternatively, expression vectors, rather than viral infections, may be used to generate the polypeptide of interest. A wide variety of expression vectors may be used, including viral vectors. The structure and use of these vectors is discussed further, below. Such vectors may significantly increase the amount of 1-CaD protein in the cells, and may permit less selective purification methods such as size fractionation (chromatography, centrifugation), ion exchange or affinity chromatograph, and even gel purification. Alternatively, the expression vector may be provided directly to target cells, again as discussed further, below.

It is expected that changes may be made in the sequence of 1-CaD while retaining a molecule having the structure and function of the natural 1-CaD, respectively. For example, certain amino acids may be substituted for other amino acids in a protein structure without appreciable loss of interactive capacity with structures such as, for example, substrate-binding regions. These changes are termed “conservative” in the sense that they preserve the structural and, presumably, required functional qualities of the starting molecule.

B. Variants

In various embodiments variants or derivatives of 1-CaD are contemplated. In particular 1-CaD variants with conservative amino acid substitutions are contemplated. Conservative amino acid substitutions generally are based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as equivalent.

In making such changes, the hydropathic index of amino acids also may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity, particularly where the polypeptide created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

Two designations for amino acids are used interchangeably throughout this application, as is common practice in the art. Alanine=Ala (A); Arginine=Arg (R); Aspartate=Asp (D); Asparagine=Asn (N); Cysteine=Cys (C); Glutamate=Glu (E); Glutamine=Gln (O); Glycine=Gly (G); Histidine=His (H); Isoleucine=Ile (I); Leucine=Leu (L); Lysine=Lys (K); Methionine=Met (M); Phenylalanine=Phe (F); Proline=Pro (P); Serine=Ser (S); Threonine=Thr (T); Tryptophan=Trp (W); Tyrosine=Tyr (Y); Valine=Val (V).

VI. Pharmaceutical Compositions

The phrases “pharmaceutically” or “pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions, vectors or cells of the present invention, its use in therapeutic compositions is contemplated. Supplementary active ingredients also can be incorporated into the compositions.

In various embodiments, the nucleic acids encoding cytoskeletal stabilizing proteins, e.g., 1-CaD and any other agents that might be delivered may be formulated and administered in any pharmacologically acceptable vehicle, such as parenteral, topical, aerosal, liposomal, nasal or ophthalmic preparations. In certain embodiments, formulations may be designed for oral, inhalant or topical administration. It is further envisioned that formulations of nucleic acids encoding cytoskeletal stabilizing proteins and any other agents that might be delivered may be formulated and administered in a manner that does not require that they be in a single pharmaceutically acceptable carrier. In those situations, it would be clear to one of ordinary skill in the art the types of diluents that would be proper for the proposed use of the polypeptides and any secondary agents required.

The active compositions of the present invention may include classic pharmaceutical preparations. Administration of these compositions according to the present invention will be via any common route so long as the target tissue or surface is available via that route. This includes oral, nasal, buccal, respiratory, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. Routes of administration may be selected from intravenous, intrarterial, intrabuccal, intraperitoneal, intramuscular, subcutaneous, oral, topical, rectal, vaginal, nasal and intraocular.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In a particular embodiment, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Culture

Cultured UMNSAH/DF-1 cells (DF-1) were purchased from American Type Culture Collection (ATTC). All cells were grown in Dulbecco's modified Eagle's medium with 10% fetal bovine serum at 39° C. and 5% CO₂. Vitrogen collagen (Type 1, bovine dermal collagen) was obtained from Celtrix Pharmaceuticals Inc. (Santa Clara, Calif.). Microelectrodes were obtained from Applied Biophysics Inc. (Troy, N.Y.), and anti-caldesmon antibody was purchased from Transduction Laboratories Inc. (LaJolla, Calif.). Oligonucleotide primers were synthesized by IDT-Technologies, Inc (Coraville, Iowa). Texas Red phalloidin and Oregon Green anti-mouse antibodies were purchased from Molecular Probes (Eugene, Oreg.). Cultured human umbilical vein endothelial cells (HUVEC) were prepared by collagenase treatment of freshly obtained human umbilical veins as previously described (Carson et al., 1989). All cells were cultured in Medium 199 (Gibco) and supplemented with 20% heat-inactivated fetal calf serum with penicillin and streptomycin. Secondary cultured porcine pulmonary artery endothelial cells (PPAEC) (passage number 4-10) were cultured in the same medium.

Cell Lysis and Total RNA Extraction

Approximately 4×10⁶ cultured HUVEC were lysed using Sigma Tri-Reagent lysis solution (Sigma-Aldrich, St. Louis, Mo.), and total RNA was extracted according to the manufacturer's instructions. Lysate was incubated at room temperature with reagent chloroform, incubated at room temperature for 15 minutes, and then centrifuged at 12,000×g for 15 minutes at 4° C. The aqueous phase was removed and mixed with isopropyl alcohol, incubated at room temperature for 10 minutes, and then centrifuged at 12,000×g for 10 minutes at 4° C. The RNA pellet was washed with 75% ethanol and then centrifuged at 7,500×g for 5 minutes at 4° C. The RNA pellet was then air dried at room temperature, resuspended in DEPC-treated water and quantitated using a spectrophotometer based on the A260/A280 ratio and by agarose, formaldehyde gel electrophoresis.

Reverse Transcription and Polymerase Chain Reaction

Five hundred nanograms of HUVEC total RNA was amplified in a one-step reverse transcriptase polymerase chain reaction using the Access RT-PCR system (Promega, Madison, Wis.). Specific primers corresponding to the full-length HUVEC 1-caldesmon were designed with BamHI restriction sites. The sense primer sequence was 5′-GGATCCATGGATGATTTTGAGCGTCG-3′ (SEQ ID NO:5) and the anti-sense primer was 5′-GGATCCAACCTTAGTGGGGGAAGTGA-3′ (SEQ ID NO:6). The final reaction contained 0.2 mM dNTP mix, 1 μM of each primer, 1 mM magnesium sulfate, 0.1-units/μl AMV reverse transcriptase, and 0.1 units/μl Tfl DNA polymerase. The reverse transcription reaction was conducted at 48° C. for 45 minutes. The polymerase chain reaction was conducted in a Biorad Gene Cycler (BioRad, Hercules, Calif.) using the following cycling conditions: a 94° C. denaturing cycle for 2 minutes; 40 cycles of 30 seconds denaturing at 94° C., 1 minute of annealing at 59° C., 7 minutes of extension at 68° C.; followed by a final extension cycle of 10 minutes at 68° C.

HUVEC 1-Caldesmon Subcloning and Adenovirus Vector Construction

The RT-PCR product was ligated overnight at 14° C. using the pGEM-T-Easy vector system according to manufacturer's instructions (Promega, Madison, Wis.). The PCR product was ligated using T4 DNA ligase. Competent DH5 α E. coli bacteria were transformed by heat shock method at 42° C. for 45 seconds followed by an ice incubation for 2 minutes. 250 μl of pre-warmed SOC (Invitrogen/Gibco BRL, Rockville, Md.) at 37° C. was then added to the mix and agitated at 250 rpm for 2.5 hrs. The transformed bacterial mixture was plated on LB-Agar plates containing 100 micrograms/μl of ampicillin. Agar plates were incubated overnight at 37° C. Positive transformants were chosen and grown in 5 ml liquid culture containing 100 μg/ml of ampicillin. One and a half ml of the culture was used to perform standard alkaline-lysis miniprep DNA isolations. Plasmid and insert orientation were examined by BamHI restriction digestion and confirmed by Sanger di-deoxy DNA sequencing. Positive transformants were then grown overnight in one liter of LB media containing 100 μg/ml of ampicillin. Plasmid DNA isolation was then performed using the Qiagen MaxiPrep Kit (Qiagen Corporation, Germany). Plasmid and insert orientation was reconfirmed using BamHI restriction digest analysis followed by DNA sequencing.

PGEM-T-1-CaD was digested using BamHI and run out on a 1% TAE agarose gel. The 1-CaD band was isolated using the BioRad Gel Slice kit (BioRad, Hercules, Calif.). The DNA insert was then ligated to pacAd5CMV, an adenovirus shuttle vector (supplied by the University of Iowa Gene Vector Core Facility) using the BamHI restriction site. Ad5 virions were produced from HEK293 cells, purified and titered by the University of Iowa Gene Vector Core Facility.

Transfection Protocol

Cultured DF-1 cells were transiently transfected with recombinant, replication-deficient adenovirions expressing wild-type 1-CaD (Ad5-CMV-1-CaD) using a CaPi coprecipitation procedure described by Fasbender (1998). Briefly, CaPi coprecipitated adenoviral particles were incubated for 1 hour by placing recombinant adenovirus particles (pfu according to the final MOI) in 1 ml of Eagle's minimal essential media at a pH of 8.0, which contained 1.8 mM Ca²⁺ and 0.86 mM inorganic phosphate. Culture medium was aspirated and the coprecipitate was exposed to cells for 30 min at room temperature. Cells were then immediately washed with fresh medium and daily for 2 days. DF-1 cells were grown in DMEM medium in 10% fetal bovine serum at 39° C. Cultured cells were transfected at doses between 10-500 MOI. After 48 hours, cells were detached with solution A (137 mM NaCl, 4.2 mM NaHCO₃, 5.4 mM KCL, 5.6 mM glucose, 0.5 mM EDTA) for different experiments.

To evaluate transfection efficiency, cultured cells were fixed with 3.7% paraformaldehyde, permeabilized with 0.1% Triton-X, and sequentially exposed to a mouse IgG anti-CaD primary antibody (Transduction Laboratories; 5 μg/ml dilution), an Oregon Green-conjugated goat anti-mouse secondary antibody (10 μg/ml) and Texas Red phalloidin (1 unit/slide). Cells were viewed using a Zeiss Axiovert 135 TV microscope, equipped with epifluorecence using rhodamine or fluorescein excitation filters and a 40×/0.75 Plan Neofluar objective. Images were captured with a 10-bit digital camera (ORCA, Hamatasu Inc.), and images were computer digitized and processed with custom algorithms written in LabView and IMAQ Vision software (National Instruments, Inc., Austin, Tex.). Groups of 100 cells that were identified by Texas Red staining of microfilaments, were evaluated for the presence of FITC-labeled 1-CaD-decorated microfilaments. The transfection efficiency was defined as the fraction of 1-CaD-decorated microfilaments to the total number of actin-stained cells.

### Example 2

### Results

1-CaD Co-Localizes with Microfilaments

Images containing Oregon Green-labeled 1-CaD filaments were assigned a green pseudocolor using Adobe Photo Shop, while images containing Texas Red labeled actin filaments were assigned a magenta pseudocolor. Images of green and magenta filters were registered, and co-localization of 1-CaD to the actin cytoskeleton was determined by the resulting formation of white or lighter-magenta colored filaments.

In vitro Wound Closure Assay to Measure Cell Motility

DF-1 cell motility was measured by wounding the cell monolayer and measuring wound closure by monitoring the traversed distance over time. DF-1 cells were plated at 70% confluence on glass cover slips coated with 100 μg/ml of fibronectin. These cells were then transfected with Ad-CaD or Ad-empty and allowed to grow to 100% confluence (2 days post-transfection). A wound was made in the monolayer using a sterile pipette tip with an outer diameter of 300 μm. The same area was captured sequentially at time intervals of 0, 1, 3, and 6 hours under phase contrast microscopy. The migrated distance was assessed at 3 locations that were separated by equal distance. Cell motility was determined as the average velocity for the 3 measurements for each time point. Replicate experiments were performed and cell motility was averaged for each time period and for each condition. Paired student t-test and analysis of variance (ANOVA) procedures were done to compare intra-subject and inter-subject differences.

Transcellular Impedance Measurements

Quantitative transcellular impedance measurements were acquired in real-time using a previously reported technique of inoculating cultured cells grown on the surface of microelectrodes coated with 100 μg/ml of fibronectin (Moy et al., 2002, Moy et al., 1998, Moy et al., 1996, Moy et al., 2000). A 1-volt, 4000 Hz AC signal was supplied through a 1-M Ω resistor to approximate a constant-current source. Voltage and phase data were measured with a SRS830 lock-in amplifier (Stanford Instruments) and then later stored and processed with a personal computer. The in-phase voltage, proportional to the resistance, and the out-of-phase voltage, proportional to the capacitive reactance, were measured. For most situations, resistance is reported because it is most sensitive to cytoskeletal-membrane properties. The resistance of the naked electrode was subtracted from the resistance of a cell-covered electrode to control for variance in biosensor properties. A uniform number of viable cells (˜650,000 cells per sample), which was determined by the amount of cells that excluded trypan blue, were inoculated on the electrode to control for the effect of cell viability. Transcellular resistance was measured as a fractional change and normalized to the cell resistance of wild-type DF-1 cells. Transcellular resistance was measured at 15 hours for inter-subject comparisons—a time point at which transcellular resistance was at steady state.

Breakdown of Cytoskeletal-Membrane Properties Using Numerical Modeling of Experimental Transcellular Impedance

A numerical analysis was used to calculate specific cell-cell and cell-substrate adhesion and membrane capacitance based on the measured transcellular impedance. The procedure is described in more detail elsewhere for endothelial cells (Moy et al., 2002, Moy et al., 2000), fibroblasts (Giaever and Keese, 1991), and for epithelial cells (Lo et al., 1995). The total impedance across a cell-covered electrode is composed of the impedance created between the ventral surface of the cell and the electrode (related to α and due to cell-matrix adhesion), the impedance created between cells (indicated by R_{b} and due to cell-cell adhesion), the transcellular impedance created from transcellular current conduction (Zₘ) and the impedance of a naked electrode (Zₙ). For these calculations, Zₘ is inversely related to membrane capacitance (Cm), which is dependent upon membrane convolution, which, in turn, is dependent on the cortical cytoskeleton. The data was expressed as a ratio of the real or imaginary measurement of cell-covered electrode to a naked electrode as a function of current frequency between 25 to 60,000 Hz. A calculated real and imaginary value was generated from the solutions of α, R_{b} and Cₘ obtained from a multi-response Levenberg-Maquardt non-linear optimization model of the real, imaginary, real and imaginary (in complex form) and real and imaginary (in magnitude form) data. An error evaluation was calculated using a Chi square analysis of the squared sum of the calculated and the experimental residuals as a function of current frequency. Solutions of α, R_{b} and Cₘ were obtained by selecting the frequency subset and Levenberg Marquardt approach that best approximated the experimental data.

Interference Reflection Microscopy (IRM)

DF-1 cells were plated at 70% confluence on glass cover slips coated with 100-μg/ml fibronectin. The next day cells were transfected with Ad-1-CaD and Ad empty. Two days post-transfection cells were fixed in PBS containing 3.7% formaldehyde for 10 minutes. A Zeiss Axiovert microscope with 63× Antiflex objective with a quarter wavelength plate was used to capture interference reflection (IR) images. A portion of the glass coverslip with the cell membrane in close contact (high refractive index) will appear darker than a section of cell-free glass. In contrast, a cell separated from the glass substrate with a thin layer of liquid will create two reflections, one between the glass-liquid and another between the liquid-cell interface, the outcome being an interference of light. The result yields varying intensities of light depending on the contact distance between the cell and the glass coverslip.

Measurement of Prestress Force:

Prestress was determined by the measured loss of constitutive isometric tension in response to cytochalasin D. Isometric tension was measured in cultured DF-1 cells inoculated on the surface of polymerized type 1 collagen membranes as described by Bodmer (1997). Isometric tension was simultaneously monitored in 2 separate isometric vectors to account for the presence of anisotropic tension. Constitutive tension was measured in cell-collagen lattices under unloaded conditions. To determine the optimal length-tension relationship during the unloaded state, the collagen gel was stretched with a micromanipulator that was attached to the transducer until a 10 mg increase in force was observed. The gel was progressively unloaded until no further decline in preload was observed. The length (Carson et al., 1989) of the gel was chosen as the no-load state. After achieving a steady-state baseline, a sham response composed of the carrier buffer for cytochalasin D was used to assess for nonspecific mechanical effects. Cells were subsequently exposed to 3 μM cytochalasin D, and the abolished constitutive tension was quantitated in real-time. The constitutive tension was defined as the measured loss in tension upon the addition of cytochalasin D.

Measurement of Actin Assembly

Actin assembly was quantitated by the fraction of insoluble actin in a Triton-X 100 based buffer as previously described by Carson and Shasby with the following modifications (Carson et al., 1992). The soluble actin fraction was precipitated by adding ice cold TCA to a final concentration of 10%, incubated on ice for 30 min., and spun for 15 min. at 14,000 rpm at 4° C. The pellet was then washed 3 times in ice-cold acetone and resuspended in 2×SDS-sample buffer to a volume equal to the final triton-insoluble fraction volume defined below. The cytoskeleton remaining on the dish was solubilized with 2% SDS, 0.5% 2-mercaptoethanol, and 100 mM NaCl; sheared through a 26-gauge needle; and centrifuged at 100,000×g for 15 minutes. The supernatant from this centrifugation was combined with the pellet saved from the Triton-soluble fraction and then an equal volume of 2×SDS sample buffer was added. This fraction was defined as the Triton insoluble fraction. Equivalent volume fractions of the soluble and insoluble fractions were subjected to electrophoresis on a 8-15% SDS polyacrylamide gradient gel, along with known concentrations of purified nonmuscle actin (Cytoskeleton Inc). The gel was stained with Coomassie blue, dried, and the actin content was quantitated by laser densitometry. The concentration of actin in each cell fraction was quantitated from an in vitro standard calibration curve of the measured radiance volume of known amounts of purified nonmuscle actin separated on the same gel.

Cell Viability Assay

Cultured cells were transfected with Ad-CaD or the controlled virus using a calcium phosphate co-precipitation technique. Cells were washed free of virus and incubated for additional 48 hours in serum containing medium. Transfected cells were exposed to trypan blue dye and the fraction of viable cells was quantitated. Viability of cells transfected with Ad-CaD and controlled virus was compared to the cell viability measured in monolayers exposed to cytochalasin for the same corresponding period.

Statistical Analysis: Data was reported as means±standard errors (S.E.). Comparisons between groups were made using an unpaired Student t-test. Comparisons between more than two groups were made using an analysis of variance (ANOVA). Individual group comparisons were done using Tukey honest significant difference test for post hoc comparison of means. Differences were considered significant at the p ≦0.05 level.

DF-1 Cells are Null for Protein and mRNA Expression of 1-CaD

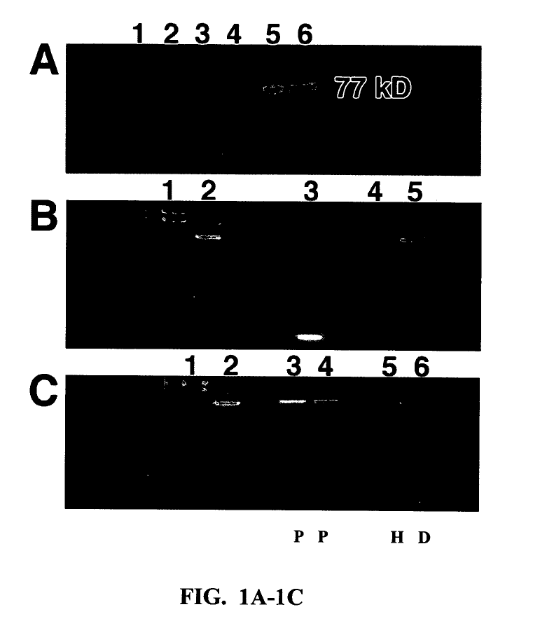

It was validated that DF-1 cells were null for 1-CaD expression. Cultured DF-1 cells were lysed with 1% SDS-sample buffer and subjected to SDS-PAGE and western blot analysis. FIG. 1A shows that DF-1 cells did not express the expected 77 Kd 1-CaD protein, while it was expressed in cultured PPAEC cells as anticipated.

DF-1 cells were null for protein expression of 1-CaD because of gene knockout from an absence of mRNA expression. RT-PCR ON isolated DF-1 total RNA was performed, and the results were compared to those of cultured PPAEC. Forward and reverse primers were designed to amplify the full-length cDNA of 1-CaD. Cultured DF-1 cells did not express the 1.6 Kd 1-CaD cDNA, while cultured PPAEC cells did (FIG. 1B). Taken together, these data demonstrate that DF-1 cells are null for 1-CaD because of an absence of 1-CaD mRNA expression. FIG. 1C illustrates the full-length cDNA of human 1-CaD, cloned from isolated total RNA obtained from cultured HUVEC, which is identical to the same size PCR fragment amplified from total RNA isolated from cultured PPAEC.

Transfection and Expression of Human 1-CaD in DF-1 Cells Using an Adenovirus Expression System

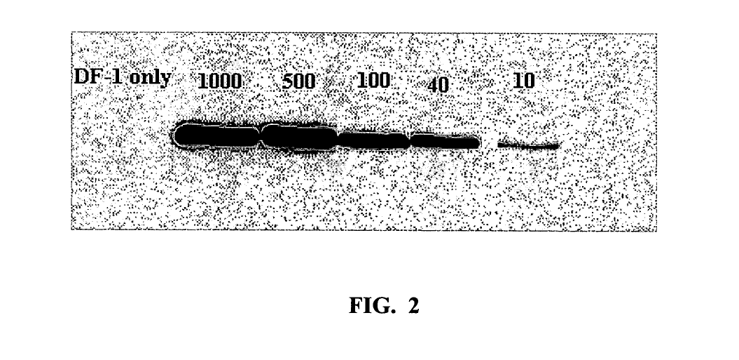

HUVEC cDNA of 1-CaD amplified by RT-PCR was subcloned into an adenovirus shuttle vector. Recombinant deficient subtype 5 adenovirus (Ad-CaD) was prepared from HEK293 cells with 1-CaD transcription controlled by a CMV promoter. Cultured DF-1 cells were transfected with Ad-CaD using a calcium-phosphate (CaPi) co-precipitation protocol as described in the Methods section. FIG. 2 shows that transfection of cultured DF-1 cells with Ad-CaD causes a dose-dependent increase in protein expression of human 1-CaD between MOI 10-1000 based on western blot analysis.

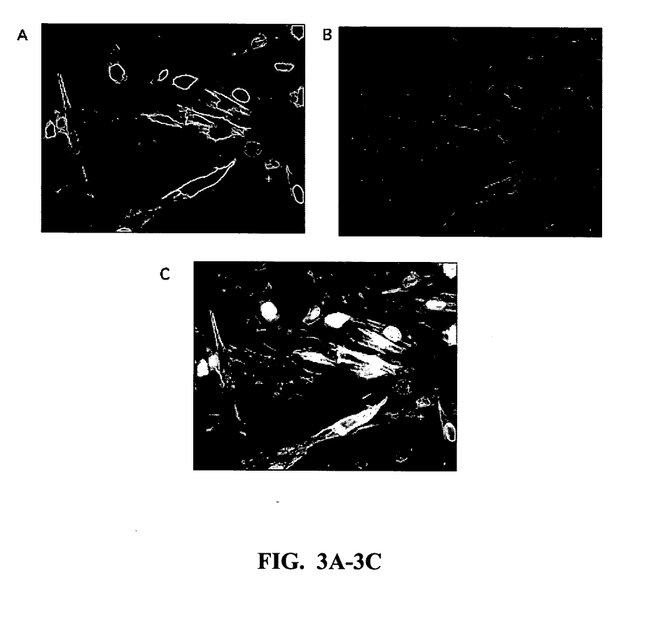

Heterologous human 1-CaD expression by CaPi co-precipitation of Ad-CaD co-localized with avian microfilaments (FIG. 3). Cells transfected with Ad-CaD were fixed, permeabilized and labeled for 1-CaD using a primary mouse antibody directed against 1-CaD and an Oregon Green conjugated secondary anti-mouse antibody. The same cells were co-stained for microfilaments with Texas Red phalloidin. Oregon green-labeled 1-CaD microfilaments were assigned a green pseudocolor, while actin filaments were assigned a magenta pseudocolor. Images were registered and superimposed. 1-CaD co-localized with the actin cytoskeleton based on the change in pseudocolor hue and saturation.

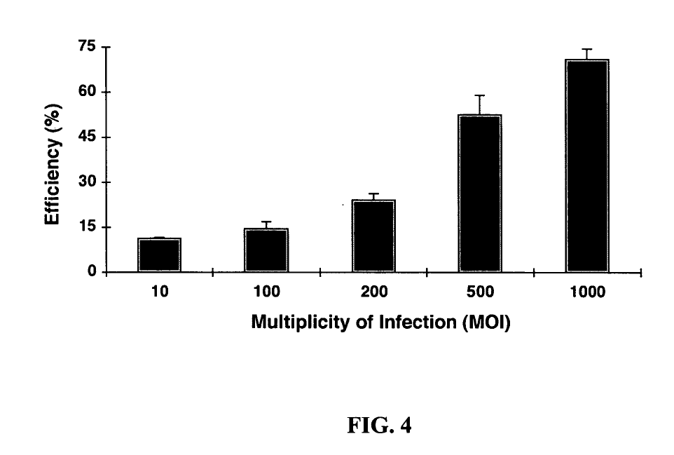

FIG. 4 shows that CaPᵢ co-precipitation of Ad-CaD mediated a dose-dependent increase in transfection efficiency in DF-1 cells. Transfection efficiency was defined as the fraction of cells that express 1-CaD co-localized filaments among the total number of cells that express avian microfilaments. Transfection efficiency increased in a dose-dependent fashion at MOI doses between 10-500. The transfection efficiency at 200 MOI was 25 percent, while it achieved a level greater than 50 percent at higher MOI doses.

The Effect of 1-CaD Expression on Resting Transcellular Resistance

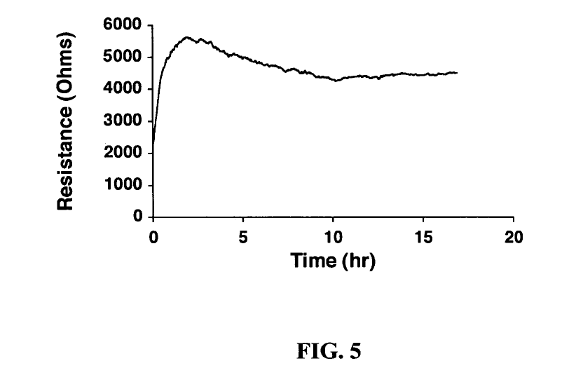

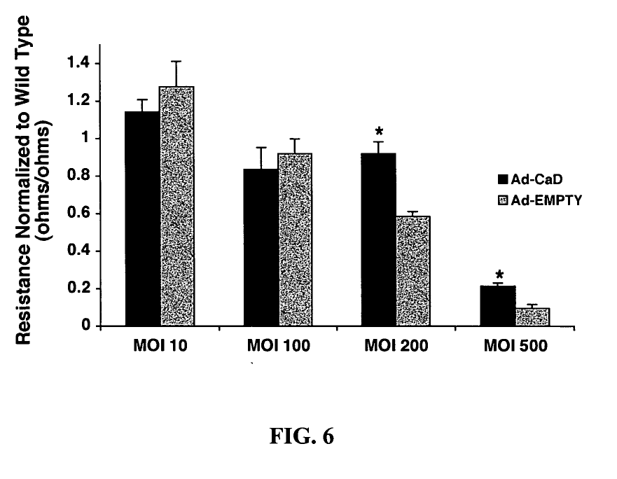

The impact of heterologous human 1-CaD expression on cytoskeletal-membrane properties in DF-1 cells was evaluated by quantifying transcellular resistance in confluent cells inoculated on a microelectrode and then applying an alternating current using a previously reported technique (Moy et al., 1996). FIG. 5 depicts a representative experiment in which transcellular resistance was monitored as confluent cells attach and spread over the microelectrode. Cell adhesion between cells transfected with Ad-CaD and Ad-only were compared at the 15 hr period, which represented a time point at which cultured monolayers achieved a steady state resistance. The resistance of the naked electrode was subtracted from the resistance of the cell-covered electrode and normalized as a fraction of transcellular resistance in wild-type cells. A dose-dependent decline in transcellular resistance in cultured cells transfected with the Ad-empty construct was observed (FIG. 6). The dose-dependent decline in resistance achieved statistical significance based on analysis of variance procedures. In contrast, Ad-CaD abrogated the dose-dependent loss in transcellular resistance in cells transfected with adenovirions. Using an unpaired student t-test, a statistical difference in transcellular resistance was obtained between cells transfected with Ad-CaD and controlled cells at dosages of 200 and 500 MOI, which represent very high adenovirus dosage. These data suggests that 1-CaD expression helps stabilizes transcellular resistance in confluent monolayers exposed to adenovirus at doses that corresponds with a transfection efficiency of 25%.

Resolving Spatial Changes in Cytoskeletal-Membrane Properties Using a Numerical Model

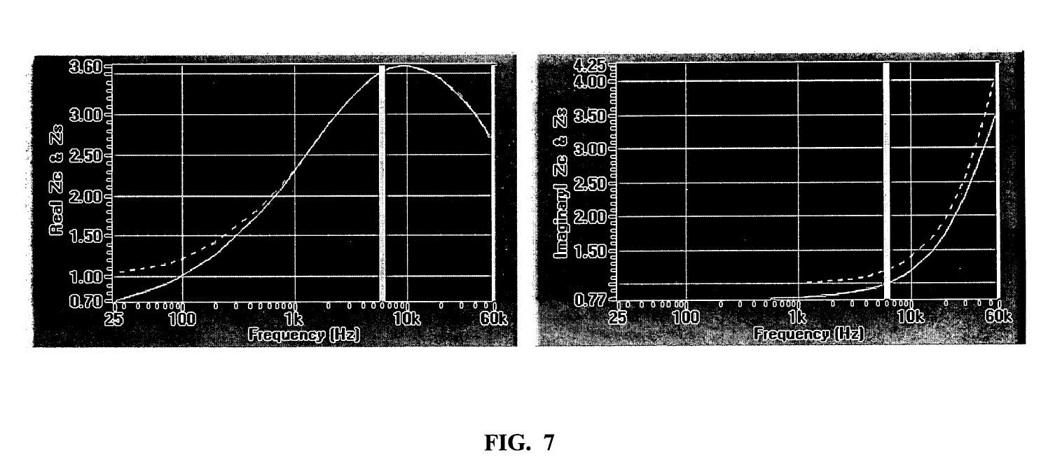

Using a previously reported mathematical model, it was next evaluated whether the difference in 1-CaD-mediated change in transcellular resistance was due to effects on cell-cell, cell-matrix adhesion, membrane capacitance or a combination of all three. Based on this model, impedance across a confluent monolayer on an electrode surface is dependent on the impedance due to cell-matrix adhesion (α), cell-cell adhesion (R_{b}) and that due to membrane capacitance (Cₘ) (Moy et al., 2002; Moy et al., 2000). A calculated resistance and capacitance was generated from iterative solutions of α, R_{b} and Cₘ obtained from a multi-response Levenberg-Maquardt non-linear optimization model of the real and/or imaginary data. FIG. 7 shows the calculated real measurements compared with the experimental measurements at frequencies between 5,000 Hz to 60,000 Hz. Chi square values were calculated to assess the potential error of the model, which represented the least square difference between the calculated and the experimental data.

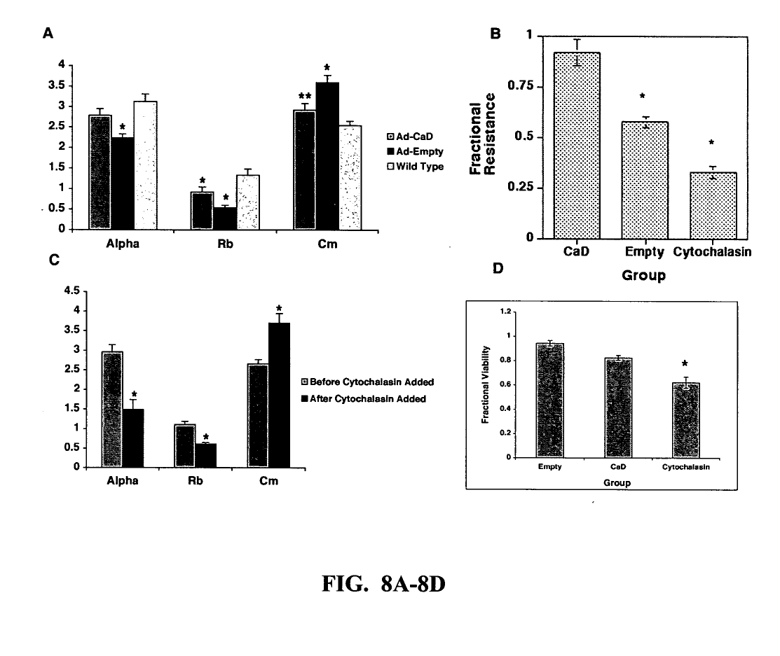

Using this method to analyze the solutions for α, R_{b} and Cₘ, the baseline effect of adenovirus exposure on α, R_{b} and Cₘ in cultured DF-1 cells was next examined (FIG. 8A). Based on this analysis, several important findings provided greater insight into the mechanisms by which adenovirus decreased transcellular resistance. First, adenovirus mediated a decrease in α and R_{b} in cultured DF-1 cells compared to wild-type cells. Cells transfected with the Ad-empty construct displayed a statistically significantly lower α and R_{b} than wild-type cells. Taken together, these data indicate that adenovirus decreases cell-cell and cell-matrix adhesion. A statistically significant increase in Cₘ in the Ad-empty transfected cells in comparison with wild-type cells was also observed. Taken together, these data demonstrate that adenovirus also decreases the experimental transcellular resistance by increasing membrane capacitance. Thus, the controlled adenovirus decreases transcellular resistance by decreasing cell-cell and cell-matrix adhesion and by increasing membrane capacitance.

FIG. 8B compares the differences in transcellular resistance between cells exposed to Ad-CaD, controlled virus and cytochalasin. Cytochalasin D mediated a greater loss in transcellular resistance than cells transfected with Ad-CaD or controlled virus at MOI of 200.

FIG. 8C compares the similarities between the effects of adenovirus on α, R_{b} and Cₘ in cultured DF-1 cells and wild-type cells exposed to cytochalasin D. Cytochalasin D mediated a decrease in α and R_{b} in cultured DF-1 cells, indicating that cytochalasin decreased cell-cell and cell-matrix adhesion. A statistically significant increase in Cₘ was also observed in response to cytochalasin D, suggesting that cytochalasin D decreased the experimental transcellular resistance by also increasing membrane capacitance. Thus, disassembly of the actin cytoskeleton decreased transcellular resistance by lowering cell-cell and cell-matrix adhesion and by increasing membrane capacitance, which was consistent with the pattern observed in cells exposed to adenovirus.

A statistically significant lower Cₘ was observed based on an analysis of variance on a Tukey multiple comparison in cells expressing 1-CaD than cells transfected with the control virus (FIG. 8A). There was no statistical difference between the Cₘ in cells expressing 1-CaD and wild-type cells, indicating that the membrane capacitance was restored to the level measured in wild-type cells. In contrast, the measured mean α between cells transfected with Ad-CaD achieved a borderline statistical significance (p=0.065) compared to cells exposed to the controlled virus. There was no statistical difference in the mean α between cells expressing 1-CaD and wild-type cells. This data suggests that there was a statistically significant borderline enhancement of cell-matrix adhesion by 1-CaD expression. The measured mean R_{b} between cells transfected with Ad-1-CaD and controlled virus were not statistically different, while there was a statistically significant difference in the mean R_{b} between cells expressing 1-CAD and wild-type cells. Taken together, these data indicate that 1-CaD expression quantitatively abrogated adenovirus-mediated loss in transcellular resistance predominately by decreasing membrane capacitance with a modest enhancing effect on cell-cell and cell-matrix adhesion.

Cells exposed to controlled adenovirus caused an increase in cell death compared to wild-type cells (FIG. 8D). Expression of wild-type 1-CaD mediated a modest additional increase in cell death than cells exposed to controlled Ad, which approach but did not achieve statistical significance (p=0.068). In contrast, exposure of cells to cytochalasin for the same time period decreased cell viability in a statistically significant manner compared to cells exposed to Ad-CaD or controlled virus.

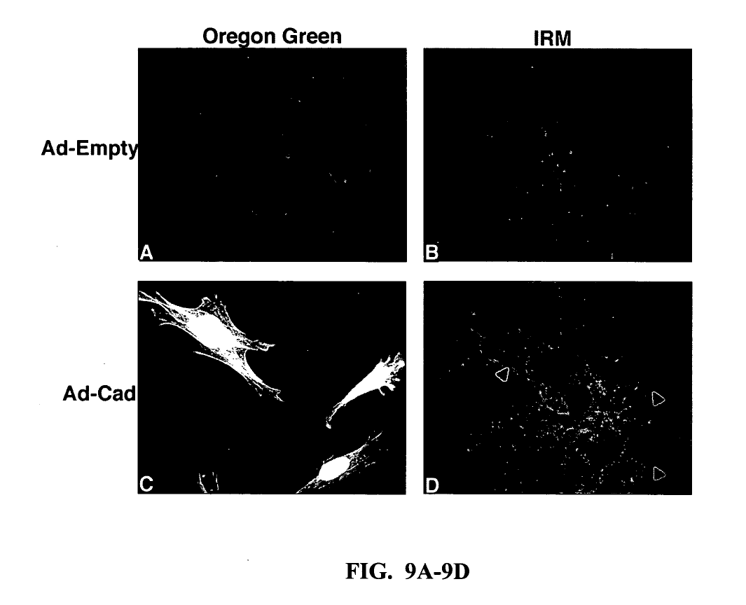

Interference reflection microscopy (IRM) was used to validate the numerical model's prediction on cell-matrix adhesion since the model predicted no significant difference in α between cells transfected with Ad-empty and Ad-1-CaD. FIG. 9 demonstrates the immunohistochemical reaction by exposing cells to a primary antibody against 1-CaD and a secondary antibody conjugated with Oregon Green (FIGS. 9A and C). The FIG. depicts cell-matrix adhesion, based on IRM (FIGS. 9B and D), in cells exposed to Ad-empty (FIGS. 9A & B) and Ad-1-CaD (FIGS. 9C & D). FIG. 9A depicts the background fluorescence of immunolabeled controlled cells, while FIG. 9B depicts the corresponding IRM image of the same cells. FIG. 9C represents cells exposed to Ad-1-CaD that demonstrate select cells expressing 1-CaD, while the remaining cells only exhibited background fluorescence. Cells expressing 1-CaD exhibited the same pattern of cell-matrix adhesion as cells exposed to Ad-1-CaD but not expressing 1-CaD. This pattern was also similar to those cells exposed to the controlled virus. This data supports the model's predictions of the effect of 1-CaD expression on cell-matrix adhesion.

1-CaD Expression and Wound Closure in Response to Mechanical Wounding

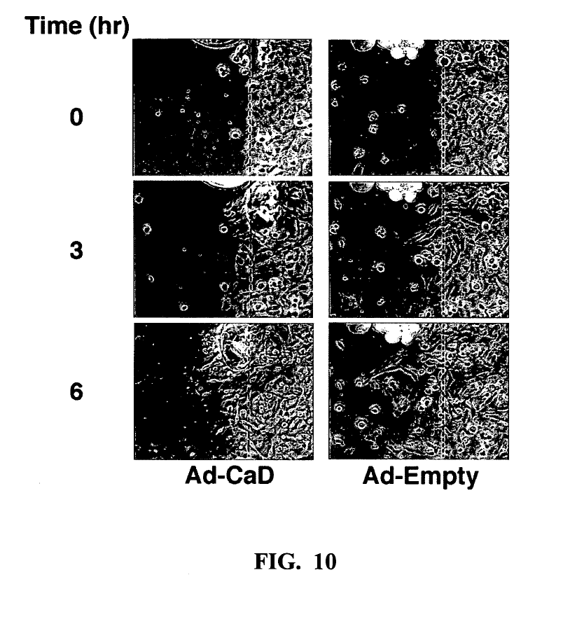

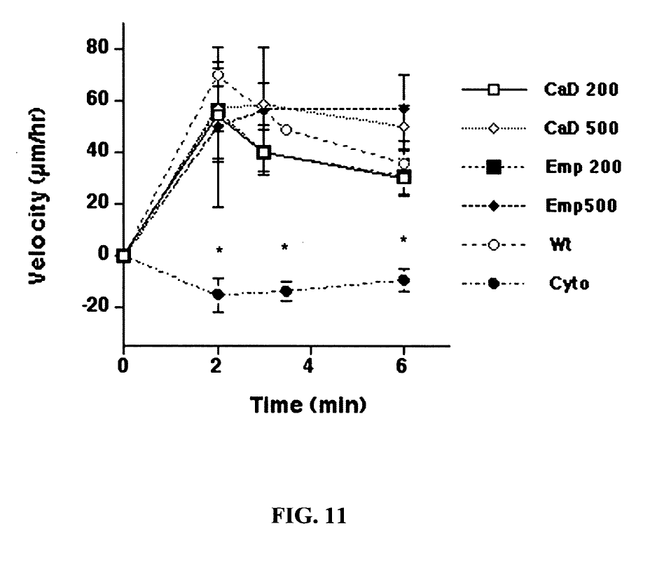

It was next evaluated whether human wild-type 1-CaD expression disturbs cytopathic effects such as wound closure. Cell motility in response to mechanical wounding was measured to address this question. Cells were transfected with either Ad-1-CaD or Ad-empty and subsequently plated on a grid coverslips. Cultured monolayers were subjected to mechanical wounding with a glass pipette and time-lapse images of cell motility were recorded over a period of several hours. Each image was equally divided into 3 regions, and the leading edge of cell migration was measured for each time point. Cell motility for each time point was determined as the average velocity measured for each of 3 regions. FIG. 10 demonstrates a representative series of images taken over several hours demonstrating cell motility of cultured cells that express 1-CAD and controlled cells. After performing replicate experiments, cell velocity was measured at each time point (FIG. 11). A statistical difference in cell velocity between cells that expressed 1-CaD and controlled cells was not observed. Additionally, this lack of difference in cell velocity was not altered even at higher transfection efficiencies. In contrast, exposure to cytochalasin completely inhibited wound repair. These data demonstrate that gene delivery of wild-type human 1-CaD does not inhibit wound repair unlike that observed in cytochalasin-treated cells.

Expression of Wild-Type Human 1-CaD does not Reduce Myosin ATPase Activity.

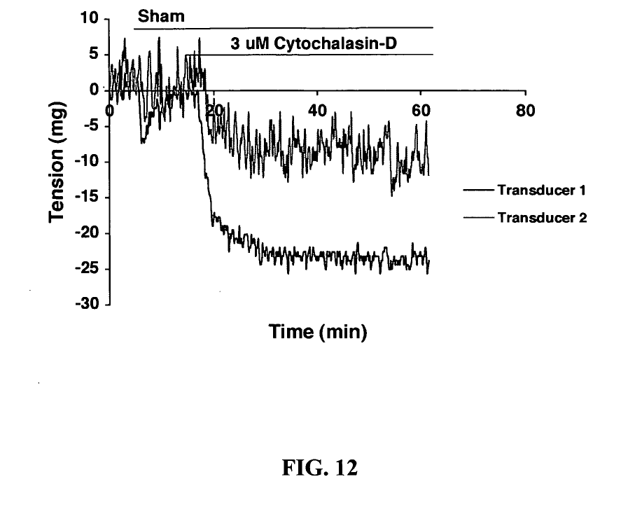

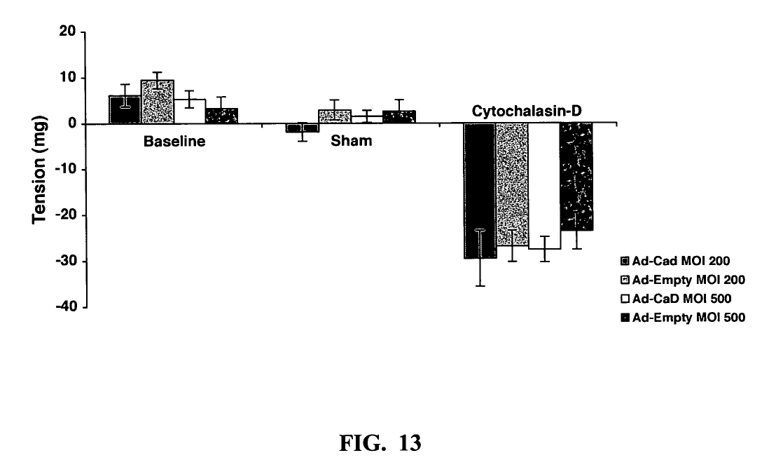

Since expression of wild-type human 1-CaD stabilizes membrane capacitance, it was next asked whether this enhancement was attributed to a reduction in myosin ATPase activity. If this hypothesis is valid, a reduction in the prestress force in cells expressing 1-CaD should be expected. To measure the resting constitutive force, the amount of tension abolished in response to cytochalasin D for cells transfected with Ad-CaD and Ad-empty was compared. Constitutive tension was measured under isometric no-load conditions in order to be consistent with the mechanical state at which transcellular resistance was measured. Each experiment required determining the initial length (Carson et al., 1989) for the no-load state. FIG. 12 shows a representative experiment in which real-time changes in isometric tension were recorded in confluent monolayers that were challenged with 3 μM cytochalasin D. After establishing a steady state baseline, each monolayer was exposed to the carrier buffer to evaluate the nonspecific mechanical effects induced by the sham response. Cells were then subsequently exposed to cytochalasin D, which abolished constitutive tension. FIG. 13 shows the changes in tension for the baseline, sham, and cytochalasin responses averaged for cells transfected with Ad-1-CaD and compared with those for cells transfected with Ad-empty. A statistical difference in tension change between cells that expressed 1-CaD and controlled cells at 200 MOI was not observed. Additionally, significant differences in myosin ATPase activity between cells that were transfected with low dose (200 MOI) and higher dose of Ad-1-CaD (500 MOI) were not observed. This data demonstrates that expression of wild-type CaD alters membrane capacitance independent of reducing myosin ATPase activity.

The Effect of Wild-Type Human 1-CaD Expression on Actin Assembly

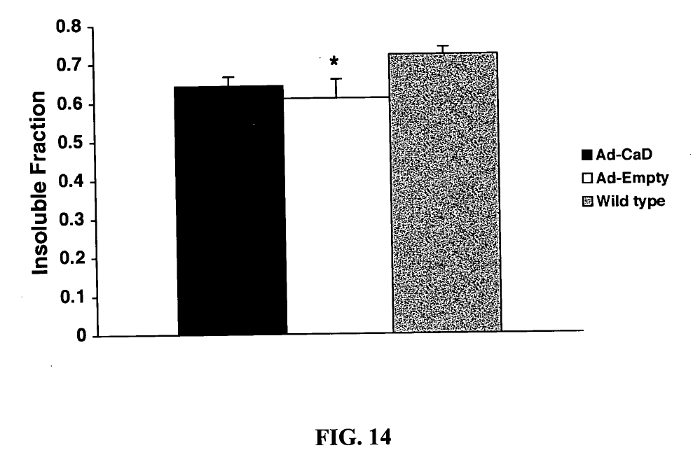

An alternative mechanism for 1-CaD to stabilize membrane capacitance is through its effects on actin assembly. Increased assembly of microfilaments could provide greater structural support on cytoskeleton-membrane interactions. This issue was addressed by measuring actin solubility in cultured cells treated with Triton-X detergents. FIG. 14 compares soluble and insoluble actin fractions between cells transfected with Ad-1-CaD and Ad-empty. Adenovirus exposure mediated a statistically significant decrease in the insoluble actin fraction from 0.72 (±0.019) in wild-type cells to 0.61 (±0.05) in cells transfected with the Ad-empty construct. However, a statistically significant difference in the insoluble actin fraction between cells transfected with the Ad-1-CaD construct and Ad-empty was not observed. These data demonstrate that expression of wild-type 1-CaD stabilizes membrane capacitance independent of effects on actin assembly, while adenovirus mediated changes in Cₘ, α and R_{b} are associated with loss in actin assembly.

### Example 3

### Lentiviral 1-CaD

The following studies examine how other caldesmon mutants may alter cell membrane integrity in DF-1 cells and in mammalian cells. Recombinant-deficient lentivirus is used instead of an adenovirus-deficient delivery system. The carboxyl terminal half of caldesmon (CaD39) is shown to protect endothelial cell membrane and the mutant, CaDS504/534E, also exhibited protective effects on cell-membrane properties in endothelial and fibroblast cells.

Generating molecular constructs of wild-type and mutant CaD: The cDNA for HUVEC 1-CaD was cloned by RT-PCR, subcloned into an adenovirus expression vector and transfected into DF-1 cells, which are null for CaD. The impact of heterologous expression of wild-type CaD on transcellular resistance in DF-1 cells was evaluated. Protocols were developed to carefully control for transfection efficiency and protein localization. Protocols were also developed that measure transcellular impedance in which the inventors correct for nonspecific viral effects and from variance in naked electrode resistance. It is demonstrated that the heterologous expression of CaD altered cell membrane properties in DF-1 cells independent of effects on actin assembly and prestress. A comprehensive number of tools to were used to evaluate the full effect of CaD in DF-1 cells. The effects of CaD on cell-membrane integrity were directed predominately on cell membrane capacitance with modest effects on cell-matrix adhesion. Biophysical-bioengineering assays are used to evaluate the impact of CaD on endothelium mechanics.

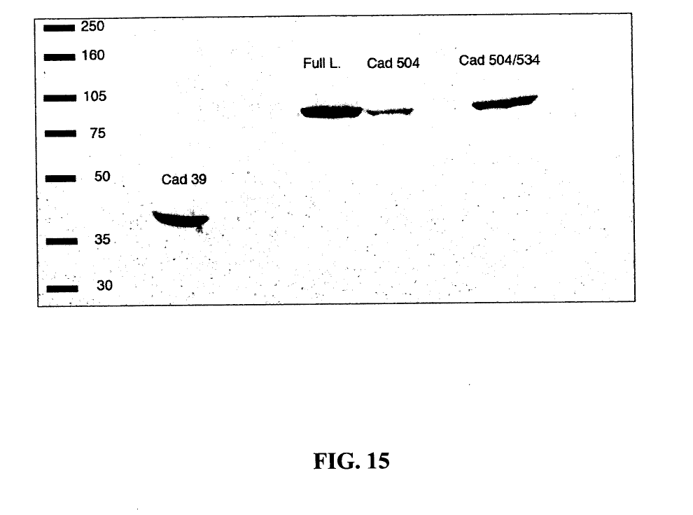

One milestone in the evaluation of the impact of CaD in cells other than DF-1 cells is the development a lentiviral expression system in which the construct containing, for example, a fusion protein with a V5 epitope at the carboxyl terminal end. FIG. 15 shows protein expression of wild-type (wt) and several CaD mutants in cultured cells by western blot procedures using an antibody against the V5 epitope. Protein expression of several CaD fusion proteins are demonstrated: expression of CaD39; expression of wt-type CaD (designated as Full L); expression of putative ERK-specific CaD mutant in which ser504 has been mutated to glutamic acid (CaDS504E or “CaD504”); and CaDS504/534E (CaD504/534) in which both ERK-specific phosphorylation sites, ser504 and ser534, have been mutated from serine to glutamic acid to produce phosphomimetic mutants.

Impact of Overexpressing Full-Length wt CaD on TER in Cultured Endothelial Cells:

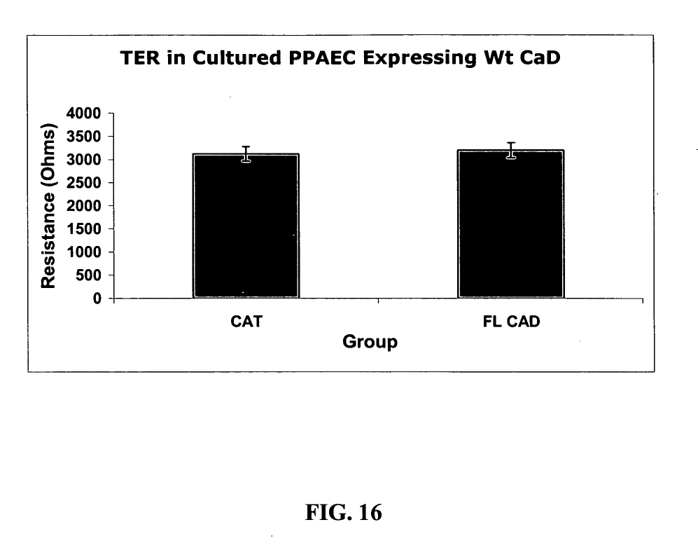

The impact of overexpressing wt-CaD in cultured porcine pulmonary artery endothelial cells (PPAEC) was determined by transfecting lentiviral constructs expressing wt-CaD and comparing the response on TER to mock transfection of the CAT gene. Transfection of wt-CaD showed no significant difference in TER in cultured PPAEC compared to monolayers that were transfected with the controlled virus (FIG. 16).

One aspect of these studies is to determine if heterologous overexpression of phosphomimetic of ERK-specific phosphorylation of 1-CaD alter constitutive and agonist-mediated barrier dysfunction.

Impact of Site-Specific Phosphorylation of ERK Phosphorylation Sites on TER.

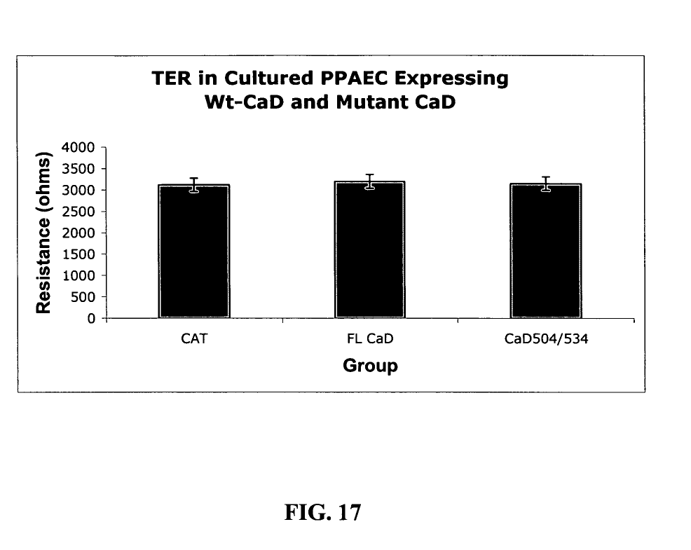

In still further studies, the impact of putative phosphorylation sites on endothelial barrier function were analyzed. To identify the impact of ERK-putative CaD phosphorylation, the inventors generated phosphomimetic mutations at the putative ERK-specific phosphorylation sites. Single and double mutations of CaD were generated at ser504 and ser534, in which serine residues have been replaced with glutamic acid residues to mimic phosphorylation. These constructs have been confirmed by sequence analysis, protein expression and packaged into lentivirus. The impact of S504E/S534E mutations on transendothelial resistance is demonstrated in cultured PPAEC by comparing mock transfection with transfection of the lentiviral construct that encodes for wt-type CaD (FIG. 17). These data indicate that ERK-specific phosphorylation is not sufficient to alter constitutive PPAEC barrier function.

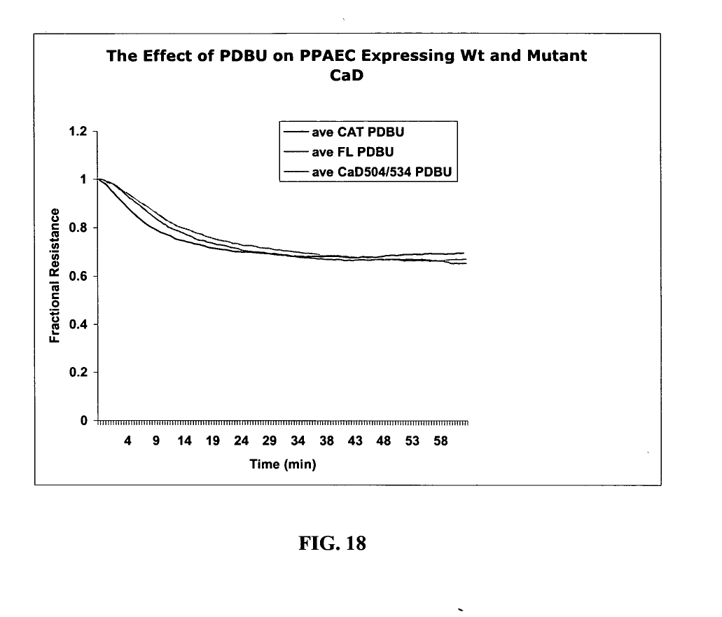

Consistent with this notion, the inventors evaluated whether phosphomimetic site-directed mutations at the putative ERK-sites are sufficient to quantitatively modify agonist-mediated barrier dysfunction in cultured PPAEC. The rationale is that if these ERK-specific phosphorylation sites were targeted by phorbol ester signal transduction and sufficient to alter endothelial barrier dysfunction, then phorbol ester stimulation would have less effect on barrier function than controlled cell. Or, if these sites were not targeted by phorbol ester signaling, but still impact actin mechanics, then the inventors would observed an augmented effect in response to phorbol ester. Monolayers transfected with S504E/S534E mutations exhibited the same fractional decrease in TER as cells transfected with wt-CaD and mocked transfected cells (FIG. 18). This preliminary data suggest that phorbol esters do not alter barrier dysfunction through ERK-specific phosphorylation of CaD.

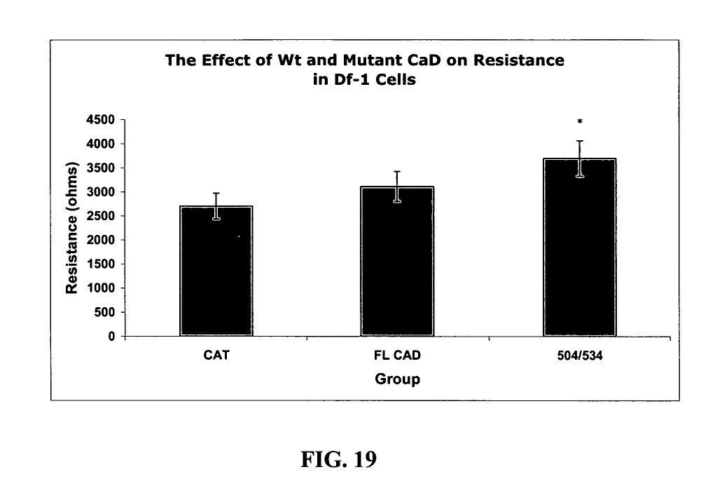

As a positive control to validate that fidelity of our ERK-specific CaD construct, the inventors performed parallel experiments in cultured DF-1 cells (FIG. 19). As shown, heterologous expression of both wt-type CaD and CaD S504/534E resulted in greater transcellular resistance than the mock transfection. Further, there was a statistical greater increase in resistance in the CaD S504/534E than cells expressing the wt-CaD. These data validates that the failure to observe a change in CaD S504/534E in cultured PPAEC was not due to a methodological problem with the mutant construct.

Taken together, these data indicate ERK-specific phosphorylation of CaD does not alter constitutive cell membrane properties in cultured PPAEC, and ERK-specific phosphorylation of CaD is not sufficient for explaining the targeted effects of phorbol ester on PPAEC barrier dysfunction.

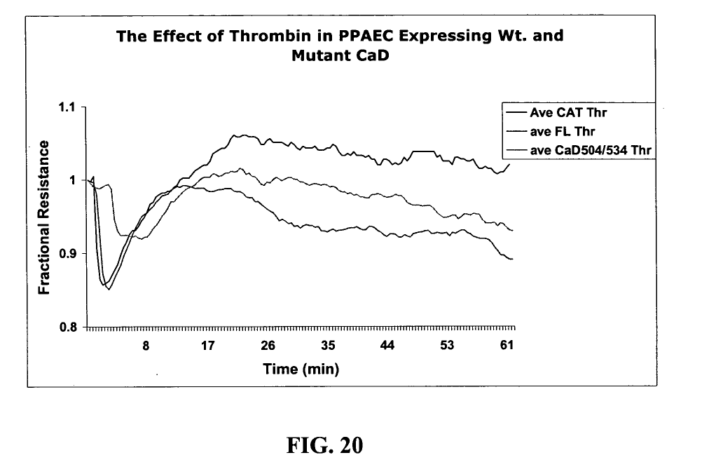

In contrast, thrombin mediated differential effects on PPAEC barrier dysfunction when wt-CaD and CaD S504/534E were overexpressed (FIG. 20). The rate and magnitude of decline in TER was unchanged between cultured monolayers transfected with the lentiviral construct that expresses wt-CaD and cells that were mocked transfected with the CAT gene. However, cells overexpressing wt-CaD exhibited a less robust recovery in resistance than mock-transfected cells in response to thrombin. In contrast, cells expressing CaD S504/534E showed a slower and smaller reduction in TER in response to thrombin (FIG. 20).

In yet further studies, the heterologous overexpression of CaD39 region was studied in regard to altering the constitutive and agonist-mediated endothelial barrier dysfunction.

The Impact of CaD39 on Cell Membrane Properties.

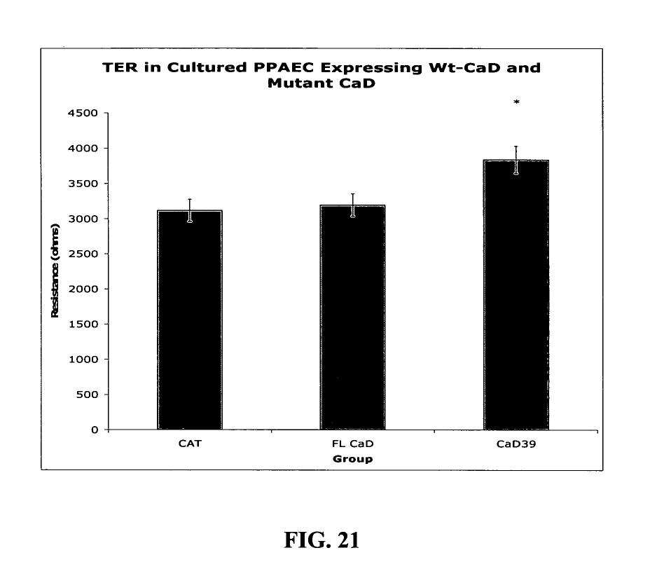

Another aspect of these studies includes the evaluation of the impact of CaD39 (actin and tropomyosin domain) on constitutive and agonist-mediated barrier dysfunction. FIG. 21 depicts the measured constitutive TER in cultured PPAEC that express heterologous CaD39, CAT (negative control) and cells expressing exogeneous wild-type CaD. Cultured PPAEC expressing CaD with deletion of the myosin-binding domain increased TER compared to cells transfected with wt-CaD and the mock transfection. These data suggest CaD39 exhibits dominant positive properties of wt CaD in the endothelium.

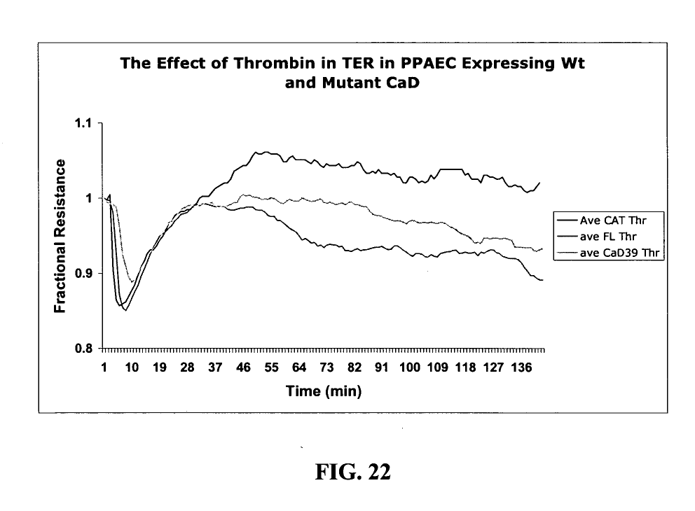

There was a slightly smaller and slower decline in thrombin-mediated TER in cells expressing the CaD39 fragment than cells transfected with the wt-CaD construct or the mock construct (FIG. 22). In contrast, the restoration of TER was attenuated by expression of the CaD39 and wt-CaD construct in thrombin-treated cultured PPAEC

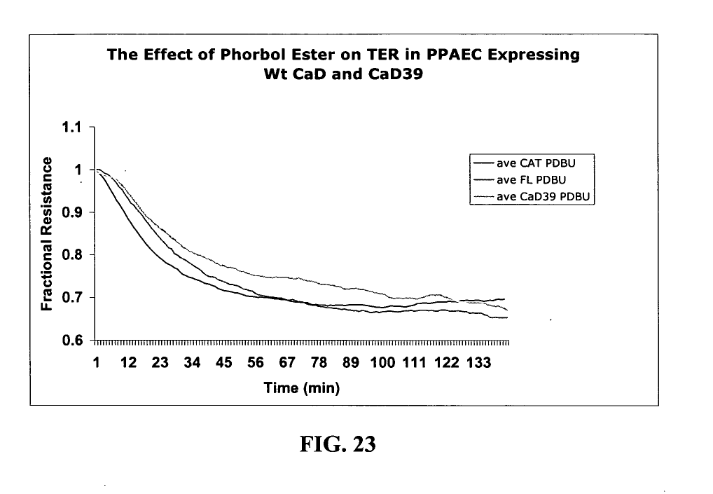

Expression of the CaD39 construct also had a small effect in attenuating the rate and amplitude of decline in TER in response to phorbol ester stimulation in cultured PPAEC (FIG. 23).

Taken together these preliminary data suggests that the CaD39 domain competes with endogeneous CaD and is more effective in increasing constitutive endothelial barrier function than the mock construct and wt-CaD in cultured PPAEC. Further, these data suggests that the CaD39 domain has a dominant positive effect over endogenous wt CaD under constitutive and agonist-mediated barrier dysfunction.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,659,774
U.S. Pat. No. 4,682,195
U.S. Pat. No. 4,683,202
U.S. Pat. No. 4,684,611
U.S. Pat. No. 4,797,368
U.S. Pat. No. 4,816,571
U.S. Pat. No. 4,879,236
U.S. Pat. No. 4,952,500
U.S. Pat. No. 4,959,463
U.S. Pat. No. 5,139,941
U.S. Pat. No. 5,141,813
U.S. Pat. No. 5,264,566
U.S. Pat. No. 5,302,523
U.S. Pat. No. 5,322,783
U.S. Pat. No. 5,384,253
U.S. Pat. No. 5,428,148
U.S. Pat. No. 5,464,765
U.S. Pat. No. 5,532,337
U.S. Pat. No. 5,538,877
U.S. Pat. No. 5,538,880
U.S. Pat. No. 5,550,318
U.S. Pat. No. 5,554,744
U.S. Pat. No. 5,563,055
U.S. Pat. No. 5,574,146
U.S. Pat. No. 5,580,859
U.S. Pat. No. 5,589,466
U.S. Pat. No. 5,591,616
U.S. Pat. No. 5,602,244
U.S. Pat. No. 5,610,042
U.S. Pat. No. 5,645,897
U.S. Pat. No. 5,656,610
U.S. Pat. No. 5,702,932
U.S. Pat. No. 5,705,629
U.S. Pat. No. 5,736,524
U.S. Pat. No. 5,739,008
U.S. Pat. No. 5,739,088
U.S. Pat. No. 5,780,448
U.S. Pat. No. 5,789,215
U.S. Pat. No. 5,871,986
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 6,602,706
U.S. Pat. No. 6,630,574
U.S. Pat. No. 6,638,502
Adelstein and Conti, Nature, 256: 597-98, 1975.
Adelstein, TIBS, 27-30, 1978.
Aksoy et al., Am. J. Physiol., 245: C255-C270, 1983.
Amano et al., J. Biol. Chem., 271: 20246-20249, 1996.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Bearer and Satpute-Krishnan, Infectious Disorders, 2: 247-264, 2002.
Belisle and Abo. J. Biol. Chem., 275: 26225-26232, 2000.
Bittner et al., Methods in Enzymol, 153: 516-544, 1987.
Blomer et al., J. Virol., 71(9): 6641-6649, 1997.
Bodmer et al., Microvascular Res., 53: 261-271, 1997.
Brown et al., J. Biol. Chem., 277: 46298-303, 2002.
Carson et al., Am. J. Physiol., 263: L664-L669, 1992.
Carson, et al., Am. J. Physiol., 257: L259-L264, 1989.
Chen and Okayama, Mol. Cell Biol., 7(8): 2745-2752, 1987.
Coomber, Exp. Cell Res., 194: 42-47, 1991.
Cotten et al., Proc. Natl. Acad. Sci. USA, 89(13): 6094-6098, 1992.
Coupar et al., Gene, 68: 1-10, 1988.
Curiel, Nat. Immun., 13(2-3): 141-164, 1994.
Daniel et al., J. Biol. Chem., 256: 7510-7514, 1981.
Eberle et al., J. Biol. Chem., 265: 16725-16728, 1990.
Eckes et al., J. Cell Science, 113: 2455-62, 2000.
Elliott and O'Hare, Cell, 88: 223-233, 1997.
EP 266 032
Fasbender et al., J. Clinical Invest., 102: 184-193, 1998.
Fechheimer et al., Proc. Natl. Acad. Sci. USA, 84: 8463-8467, 1987.
Fishkind et al., J. Cell Biology, 114: 967-975, 1991.
Fraley et al., Proc. Natl. Acad. Sci. USA, 76: 3348-3352, 1979.
Friedmann, Science, 244: 1275-1281, 1989.
Froehler et al., Nucleic Acids Res., 14(13): 5399-5407, 1986.
Galazkiewicz et al., Europ. J. Biochem., 181: 607-614, 1989.
Gandhi et al, Amer. J. Physiology, 272: F214-21, 1997.
Giaever and Keese, Proc. Natl. Acad. Sci. USA, 88: 7896-7900, 1991.
Gopal, Mol. Cell Biol., 5: 1188-1190, 1985.
Graham and Van Der Eb, Virology, 52: 456-467, 1973.
Grinnell. J. Cell. Biol., 124: 401-404, 1994.
Grunhaus et al., Seminar in Virology, 200(2): 535-546, 1992.
Harland and Weintraub, J. Cell Biol., 101(3): 1094-1099, 1984.
Hayashi et al., Proc. Nat. Acad. Sci. USA, 89: 12122-12126, 1992.
Hecht et al., Amer. J. Physiology, 271: C1678-84, 1996.
Helfman et al., Molecular Biol. Cell, 10: 3097-112, 1999.
Herz and Roizman, Cell, 33(1): 145-151, 1983.
Holwell et al., J. Cell Science, 110: 1947-56, 1997.
Horwich et al. J. Virol., 64: 642-650, 1990.
Hosoya et al., J. Cell Biology, 121: 1075-82, 1993.
Huber, Int. J. Biochem. Cell Biol., 29(8-9): 1047-1051, 1997.
Humphrey et al., Gene, 112: 197-204, 1992.
Imaizumi et al., J. Dermatol. Science, 11: 134-41, 1996.
Ishikawa et al., FEBS Letters, 299: 54-56, 1992.
Iyengar et al., J. Virology, 72: 5251-5255, 1998.
Jackson and Bellett, J. Virology, 63: 311-318, 1989.
Kaeppler et al., Plant Cell Reports, 9: 415-418, 1990.
Kaneda et al., Science, 243: 375-378, 1989.
Kato et al, J. Biol. Chem., 266: 3361-3364, 1991.
Kawamoto et al., J. Biol. Chem., 264: 2258-2265, 1989.
Kelleher and Vos, Biotechniques, 17(6): 1110-7, 1994.
Kimura et al., Science, 273: 245-248, 1996.
Kyte and Doolittle, J. Mol. Biol., 157(1): 105-132, 1982.
Lash et al., J. Biol. Chem., 261: 16155-60, 1986.
Laughlin et al., J. Virol., 60(2): 515-524, 1986.
Lebkowski et al., Mol. Cell. Biol., 8(10): 3988-3996, 1988.
Leopold et al., Human Gene Therapy, 11: 151-65, 2000.
Li et al., J. Virology, 72: 8806-12, 1998.
Lo et al., Biophysical J., 69: 2800-2807, 1995.
Macejak and Sarnow, Nature, 353: 90-94, 1991.
Mak et al., J. Biol. Chem., 266: 6678-81, 1991.
Makuch et al., Biochimica et Biophysica Acta, 1206: 49-54, 1994.
Mann et al., Cell, 33: 153-159, 1983.
Marston and Redwood, Biophysical J., 279: 1-16, 1991.
Matsumura and Yamashiro, Current Opinion Cell Biol., 5: 70-6, 1993.
McLaughlin et al., J. Virol., 62(6): 1963-1973, 1988.
Miller et al., Am. J. Clin. Oncol., 15(3): 216-221, 1992.
Montesano and Orci, Proc. Natl. Acad. Sci. USA, 85: 4894-4897, 1988.
Moy et al., Am. Journal of Physiol., 274: L1024-L1029, 1998.
Moy et al., Am. Journal of Physiol., 282: H21-H29, 2002.
Moy et al., J. Clin. Invest., 92: 1198-1206, 1993.
Moy et al., J. Clin. Invest., 97: 1020-1027, 1996.
Moy et al., Lung Cellular & Molecular Physiology, 278: L888-98, 2000.
Muzyczka, Curr. Topics Microbiol. Immunol., 158: 97-129, 1992.
Nabel et al., Science, 244(4910): 1342-1344, 1989.
Naldini et al., Science, 272(5259): 263-267, 1996.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721: 185-190, 1982.
Nicolau et al., Methods Enzymol., 149: 157-176, 1987.
Omirulleh et al., Plant Mol. Biol., 21(3): 415-28, 1993.
Paskind et al., Virology, 67: 242-248, 1975.
PCT Appln. WO 94/09699
PCT Appln. WO 95/06128
Pelletier and Sonenberg, Nature, 334(6180): 320-325, 1988.
Pilcher et al., Plastic & Reconstructive Surgery, 96: 1188-1195. 1995.
Potrykus et al., Mol. Gen. Genet., 199: 183-188, 1985.
Potter et al., Proc. Natl. Acad. Sci. USA, 81: 7161-7165, 1984.
Remington's Pharmaceutical Sciences, 15^{th} ed., pages 1035-1038 and 1570-1580, Mack Publishing Company, Easton, Pa., 1980.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10: 689-695, 1990.
Roux et al., Proc. Natl. Acad. Sci. USA, 86: 9079-9083, 1989.
Sambrook et al., In: Molecular cloning, Cold Spring Harbor Laboratory Press, Cold Spring Harbor, N.Y., 2001.
Schuppan et al., Zeitschrift fur Gastroenterologie, 33: 546-50, 1995.
Singer, Am. J. Physiol., 259: C631-C639, 1990.
Sobue and Sellers, J. Biol. Chem., 266: 12115-12118, 1991.
Staufenbiel et al., J. Virology, 60: 1186-91, 1986.
Surgucheva and Bryan, Cell Motility Cytoskeleton, 32: 233-243, 1995.
Tanaka et al., European J. Biochem., 188: 495-500, 1990.
Temin, In: Gene Transfer, Kucherlapati (Ed.), NY, Plenum Press, 149-188, 1986.
Tratschin et al., Mol. Cell. Biol., 4: 2072-2081, 1984.
Tur-Kaspa et al., Mol. Cell Biol., 6: 716-718, 1986.
van Loo et al., J. Virology, 75: 961-970, 2001.
Warren et al., Cell Motility Cytoskeleton, 34: 215-29, 1996.
Warren et al., J. Cell Biology, 125: 359-68, 1994.
Wilson et al., Science, 244: 1344-1346, 1989.
Wong et al., Gene, 10: 87-94, 1980.
Wu and Wu, Biochemistry, 27: 887-892, 1988.
Wu and Wu, J. Biol. Chem., 262: 4429-4432, 1987.
Yamakita et al., J. Biological Chemistry, 267: 12022-9, 1992.
Yamashiro et al., Molecular Biology Cell, 12: 239-50, 2001.
Yamashiro et al., Nature, 344: 675-678, 1990.
Yamashiro-Matsumura and Matsumura, J. Cell Biology, 106: 1973-83, 1988.
Zufferey et al., Nat. Biotechnol., 15(9): 871-875, 1997.

1. A pharmaceutical composition comprising a mammalian viral expression vector encoding 1-caldesmon (1-CaD) in a pharmaceutically acceptable carrier.

2. The method of claim 1, wherein the mammalian viral expression vector is an adenoviral, lentiviral, HSV, AAV, MMLV, or vaccinia vector.

3. The composition of claim 2, wherein the mammalian viral expression vector is an adenoviral expression vector.

4. The composition of claim 3, wherein the adenoviral expression vector encodes a replication competent, a conditionally replication competent or a replication defective adenovirus.

5. The composition of claim 3, wherein the adenoviral expression vector lacks all or part of an E1 coding sequence.

6. The composition of claim 3, wherein the adenoviral expression vector encodes an adenovirus with an altered tropism.

7. The composition of claim 1, wherein the mammalian viral expression vector encodes at least a second therapeutic protein.

8. The composition of claim 1, further comprising at least one pharmaceutically acceptable excipient.

9. The composition of claim 1, wherein the composition is capable of being injected, administered topically, or nebulized.

10. An isolated polynucleotide comprising a nucleic acid sequence encoding a polypeptide as set forth in SEQ ID NO:2.

11. The polynucleotide of claim 10, further comprising a promoter sequence.

12. The polynucleotide of claim 11, further comprising a polyadenylation signal.

13. The polynucleotide of claim 10, wherein the polynucleotide is comprised in a viral vector.

14. The polynucleotide of claim 13, wherein the viral vector is an adenoviral vector.

15. The polynucleotide of claim 14, wherein the adenoviral vector encodes a replication competent, a conditionally replication competent or a replication defective adenovirus.

16. A method comprising administering an effective amount of an expression vector encoding 1-CaD to a subject infected with a pathogen.

17. The method of claim 16, wherein the subject is an animal.

18. The method of claim 16, wherein the subject is a human.

19. The method of claim 16, wherein the pathogen is a viral pathogen.

20. The method of claim 16, wherein the expression vector is an adenoviral vector.

21. The method of claim 20, wherein approximately 102 to 1015 plaque forming units of adenovirus/kg body weight are administered.

22. The method of claim 16, wherein the expression vector is administered in a single dose.

23. The method of claim 16, wherein the expression vector is administered in more than one dose.

24. The method of claim 16, wherein administration of the expression vector is by inhalation.

25. A method for attenuating a viral infection comprising administering to a subject having a viral infection an effective amount of an expression cassette encoding 1-CaD.

26. The method of claim 25, wherein the subject is immunocompromised.

27. The method of claim 25, wherein the expression cassette is comprised in a viral vector.

28. The method of claim 27, wherein the viral vector is an adenoviral vector.

29. The method of claim 25, further comprising administrating at least a second antiviral composition.

30. The method of claim 29, wherein the second antiviral composition is interferon, nucleoside analogs, cytosine-arabinoside, adenine-arabinoside, iodoxyuridine or acyclovir.

31. The method of claim 25, wherein administering is by inhalation.

32. The method of claim 25, wherein the viral infection is a respiratory infection.

33. The method of claim 32, wherein the respiratory infection is an adenovirus infection.

34. A prophylactic method comprising administering an effective amount of an expression vector encoding 1-CaD to a subject at risk of infection by a pathogen.

35. The method of claim 34, wherein the expression vector is an adenoviral expression vector.

36. The method of claim 34, wherein administration of the vector is by inhalation.

37. The method of claim 34, wherein the pathogen is a viral pathogen.

38. The method of claim 35, wherein approximately 102 to 1015 plaque forming units of adenovirus/kg body weight are administered.

39. The method of claim 34, wherein the vector is administered in a single dose.

40. The method of claim 34, wherein the vector is administered in more than one dose.
